(19) United States
(12) Patent Application Publication
(10) Pub. No.: US 2020/0196881 A1
(43) Pub. Date: Jun. 25, 2020

(54) METHODS, DEVICES AND MACHINE READABLE PROGRAMS FOR CUFF-LESS BLOOD PRESSURE MEASUREMENT

(71) Applicant: Marc Zemel, Valhalla, NY (US)

(72) Inventor: Marc Zemel, Valhalla, NY (US)

(21) Appl. No.: 16/809,902

(22) Filed: Mar. 5, 2020

Related U.S. Application Data
(63) Continuation of application No. PCT/US2018/049781, filed on Sep. 6, 2018.
(60) Provisional application No. 62/555,045, filed on Sep. 6, 2017.

(51) Int. Cl. A61B 5/022; A61B 5/0404; A61B 5/024; A61B 5/00

(52) U.S. Cl. CPC A61B 5/02225; A61B 5/0404; A61B 5/02444; A61B 5/0024; A61B 2562/0247; A61B 5/6815; A61B 5/6824; A61B 5/02416; A61B 5/6819

(57) ABSTRACT

Methods, devices and machine readable programs are presented for measuring blood pressure using a device configured to guide a user regarding placement of an anatomical region of interest in relation to a sensor. The methods can includes measuring, by the sensor, a pressure applied to the sensor and measuring, by the sensor, blood volume oscillations in the anatomical region of interest while pressure is being applied to the sensor by the anatomical region of interest. The methods can further include estimating, by processing circuitry (e.g, computer processor(s), memory and the like) of the device, a blood pressure value for the user from the measured pressure and the measured blood volume oscillations.

### CROSS-REFERENCE TO RELATED APPLICATIONS

The present application is a continuation of and claims the benefit of priority to International Application No. PCT/US2018/049781, filed Sep. 6, 2018, which in turn claims the benefit of U.S. Provisional Application No. 62/555,045, filed Sep. 6, 2017. Each of the foregoing patent applications is hereby incorporated by reference herein in its entirety for any purpose whatsoever.

### BACKGROUND OF THE DISCLOSURE

### Field

The present disclosure relates to methods and devices for cuff-less blood pressure measurement.

### Description of Related Art

Hypertension afflicts about one-fourth of the world's adult population. It is a major risk factor for stroke and heart disease and is therefore a “silent killer”. Hypertension can be treated with lifestyle changes and medication. Medical therapy is associated with a 35-40% reduction in the risk of stroke and a 15-25% reduction in the risk of heart disease. Hence, hypertension management is an archetypical example of preventive, proactive healthcare. However, the detection of high blood pressure (BP) is often missed. An estimated 20% of people with hypertension in the US do not know they have it. Further, BP in known hypertensive patients is often uncontrolled. An estimated 53% of hypertensive patients in the US do not have their BP under control.

Hypertension detection and control rates are much worse elsewhere, especially in low resource settings wherein personnel trained in BP measurement and the means for people to have their BP measured are lacking. Hypertension management is complicated by the well-known masked and white coat effects in the clinic and large BP variability amongst few measurements. In fact, ambulatory BP monitoring is now considered the gold standard for the diagnosis of high BP. Ubiquitous BP monitoring technology can improve hypertension detection by providing serial, out-of-clinic measurements in the mass population and can enhance hypertension control by providing continual feedback to the individual patient.

Several methods are available for measuring BP. However, none of these methods offers ubiquitous BP monitoring capabilities. Catheterization is the gold standard method. This method measures a BP waveform by placing a strain gauge in fluid contact with blood. However, this method is invasive. Auscultation is the standard clinical method. This method measures systolic BP (SP) and diastolic BP (DP) by occluding an artery with a cuff and detecting the Korotkoff sounds using a stethoscope and manometer during cuff deflation. The first sound indicates the initiation of turbulent flow and SP, while the fifth sound is silent and indicates the renewal of laminar flow and DP. The method is non-invasive but requires a skilled operator. Further, due to safety and ecological concerns, mercury manometers are being replaced with high maintenance aneroid manometers.

Oscillometry is the most popular non-invasive and automatic method. This method measures mean BP (MP), SP, and DP using an inflatable cuff with a sensor to record the pressure inside it. The recorded cuff pressure not only rises and falls with cuff inflation and deflation but also shows tiny oscillations indicating the pulsatile blood volume in the artery. The amplitude of these oscillations varies with the cuff pressure, as the arterial blood volume-transmural pressure relationship is nonlinear. Transmural pressure of an artery is defined as the internal pressure (i.e., BP) minus the external pressure (cuff pressure in this case). The BP values are estimated from the oscillogram (i.e., the oscillation amplitudes versus the cuff pressure) using an algorithm (e.g., fixed-ratios). However, automatic cuffs do not afford ubiquitous BP monitoring capabilities. That is, people in low resource settings may not have any access to such devices; others must go out of their way (e.g., to a pharmacy) to use these devices; and even people who own a device cannot carry and use them outside their homes.

Volume clamping is a non-invasive and automatic method used in research. This method measures a finger BP waveform by using a cuff with a photoplethysmography (PPG) sensor built-in to measure the blood volume. The blood volume at zero transmural pressure is estimated by slowly varying the cuff pressure. The cuff pressure is then continually varied to maintain this blood volume throughout the cardiac cycle via a fast servo-control system. The applied cuff pressure may thus equal BP. However, in addition to requiring a cuff, the method is prohibitively expensive.

Tonometry is another research method. This method measures a BP waveform by pressing a manometer-tipped probe on an artery. The probe must flatten or applanate the artery so that its wall tension is perpendicular to the probe. However, manual and automatic applanation have proven difficult. As a result, while the method should not require any calibration, the measured waveform has been routinely calibrated with a cuff in practice. Furthermore, the method is likewise costly. As a result, cuff-less BP monitoring technology is being widely pursued.

Much of these efforts are based on the principle of pulse transit time (PTT). PTT is the time delay for the pressure wave to travel between two arterial sites. An increase in BP causes the arteries to stiffen which, in turn, causes PTT to decline. So, PTT is often inversely correlated with BP in individual subjects. Further, PTT may be simply determined from the relative timing between proximal and distal arterial waveforms.

Hence, PTT carries the advantage of possibly offering passive BP monitoring without using a cuff. However, this approach also has major disadvantages. Firstly, PTT not only changes with BP but also smooth muscle contraction (especially when measured in small arteries) and aging and disease (especially when measured in large arteries). Smooth muscle contraction occurs acutely and thus severely limits the accuracy of the approach, whereas aging and disease are longer processes that prevent PTT from being able to track chronic changes in BP such as the common development of isolated systolic hypertension due to large artery stiffening with aging. Secondly, the required calibration of PTT in units of msec to BP in units of mmHg must either be population-based and thus error-prone or involve periodic use of a BP cuff and thus not truly cuff-less.

In sum, hypertension is a major cardiovascular risk factor that is treatable, yet high BP detection and control rates are unacceptably low. Ubiquitous BP monitoring technology can improve hypertension management, but oscillometric and other available non-invasive BP measurement devices employ an inflatable cuff and therefore do not afford such monitoring capabilities. While the PTT approach can potentially permit cuff-less and passive BP monitoring, its accuracy will be limited due to confounding physiology and the need for calibration. Hence, there is a need in the art for a ubiquitous method for reliable, cuff-less measurement of BP.

### SUMMARY OF THE DISCLOSURE

The present disclosure concerns improvements with respect to the pressure sensor and blood volume sensor described in PCT/US17/20739.

In some implementations, the present disclosure applies the pressure sensor and blood volume sensor described in PCT/US17/20739 to arteries in the body other than the finger. The present specification thus includes much of the description of PCT/US17/20739 that is enhanced in certain pertinent respects in addition to the novel methods provided herein, as will be appreciated by those of skill in the art.

In the disclosed methods, the device of PCT/US17/20739 is pressed by the user, or by an automatic or automated device, against a target artery in the anatomical region of interest. For example, such arteries can include the superficial and deeper arteries of any part of the body including the digital and other arteries of the hand, thumb, nose (inside or outside), forehead, scalp, ear, toe, wrist (radial artery), arm (brachial, axillary, subclavian, or ulnar), lower extremities (femoral, popliteal, tibial, or dorsalis pedis), or the trunk (aorta or ileac). In order to measure the blood pressure, one of the following alternate steps is preferably performed.

For example, in a first implementation of a method in accordance with the present disclosure, a user can locate the artery and then apply the device to the artery.

If desired, in a further implementation, the user can locate the general area on the body where the artery should be, apply the device to the target area and attempt to make a measurement. The device can then provide feedback on whether the measurement produced a reading of sufficient quality. If the reading is not sufficient, not, the user can re-position the device and make another attempt. The device may have guiding features (mechanical or other) that can be used to assist the user in the proper placement of the device with respect to the target area.

If desired, in still a further implementation, the device can be applied to the body, can automatically apply pressure required to make a measurement, and adjust its position if necessary to improve the signal quality. For example, the finger can be placed in the device and a mechanical (e.g., pneumatic, or electromechanical) mechanism can be actuated which presses the finger against the disclosed pressure sensor and blood volume sensor combination.

If desired, in still a further implementation, an array of combinations of pressure and blood volume sensors combinations can be placed around the target anatomical region of interest and then measurements can be taken from each of the sensors within the array. Appropriately configured software can then process signal information relating to readings that are most likely to produce the most accurate measurement of the blood pressure based on pre-defined criteria for the signals, such as signal strength, signal-to-noise ratio, other waveform characteristics (amplitude, frequency, etc.), and the like.

It will also be appreciated that the methods and systems can include a hard wired or wireless (e.g., Bluetooth) device that includes a pressure sensitive surface that more precisely conforms to, or is better configured to detect, pressure readings at an anatomical location. For example, and external sensor that is convex can be provided that communicates with the smart phone to obtain a signal reading. The convex surface can be a plane curved along one dimension, or two dimensions, as desired. Alternatively a curved portion of a smart phone screen (e.g., a curved edge region of the screen) can be used to detect pressure signals at an anatomical location that is best suited for such a geometry.

When the target anatomical region of interest is not at heart level, the measured blood pressure can be expected to be different than the brachial blood pressure, which is conventionally measured with an arm cuff and upon which most common clinical decisions relating to blood pressure are based. In this instance, there are two differences—one due to hydrostatic bias when the target anatomical region of interest is not at the level of the heart and one due to differences in the internal pressure and blood flow characteristics of the arterial tree within the individual. The hydrostatic bias can be corrected by having the user hold the target anatomical region of interest, such as the wrist, at heart level, by having the device measure the height difference between the measurement location and the heart, by using a nomogram to allow the approximate graphical computation of the blood pressure that estimates the height difference based on the name of the measurement location, or by having the user input the height difference manually, for example. Measurement of the distance above or below the heart can, if desired, be accomplished using a camera to take a picture of the subject showing the measurement location and computing the distance, and/or by holding an accelerometer at the nominal heart location and saving this reference location and then calculating the vertical displacement from the heart using accelerometer data during measurement. A GPS sensor can be similarly employed for this purpose. Machine readable software instructions and supporting circuitry that are suitably configured with algorithms based on appropriate mathematical models can then be used to correct the blood pressure values based on the vertical displacement from the heart.

The differences in the blood pressure readings arising from the differences in the internal blood pressure and flow characteristics of the subject can be corrected through the use of a transformation function software and/or circuitry. For example, an illustrative device can include a library of transformation functions, such as from the finger to the arm, from the wrist to the arm, and others. Such transformation functions are known in the literature or can be empirically provided that can be based on the location of the target anatomical region of interest (either input by the user or automatically detected by the device via a camera or other sensor), The device can be configured and programmed to select the most relevant transformation function to correct the blood pressure reading accordingly.

The pressure and blood volume sensor construction nominally has a 10 millimeter contact area for the target body part (such as the finger). Other shapes and sizes are possible, such as ellipses, rectangles, squares, having areas ranging from about 5 mm² to about 500 mm², and in any desired increment therebetween of 1 mm².

The blood volume sensor, such as the photoplethysmograph (PPG) sensor, typically comprises an LED that emits either near infrared or visible light (such as green, yellow or red) emission and a photodetector. The separation between the photodetector and the LED ranges from 1 to 10 mm and in any desired increment therebetween of 0.1 mm. The area of the photodetector ranges from 1 mm² to 250 mm² and in any desired increment therebetween of 1 mm².

In some implementations, the disclosure provides methods of measuring blood pressure using a handheld user device that includes detection, processing and display circuitry. Some embodiments of the methods include guiding a user regarding placement of an anatomical region of interest in relation to a sensor of the handheld user device, measuring, by the sensor, a measured pressure applied to the sensor, measuring, by the sensor, measured blood volume oscillations in the anatomical region of interest while pressure is being applied to the sensor by the anatomical region of interest, estimating, by the processing circuitry of the device, a blood pressure value for the user from the measured pressure and the measured blood volume oscillations, and generating a signal to present the blood pressure value.

If desired, the anatomical region of interest can include, for example, one or more of a superficial artery, a non-superficial artery, a digital artery other than the transverse palmer arch artery, an artery of the hand other than the transverse palmer arch artery, an artery of the thumb, an artery of the nose, an inner artery of the nose, an outer artery of the nose, an artery of the forehead, an artery of the scalp, an artery of the ear, an artery of the toe, an artery of the wrist, the radial artery, the brachial artery, the axillary artery, the subclavian artery, the ulnar artery, the femoral artery, the popliteal artery, the tibial artery, the dorsalis pedis artery, the aorta, and the iliac artery.

If desired, the method can additionally or alternatively include placing a visual indicator on an exterior surface of the device, wherein the visual indicator is arranged in relation to the sensor and the sensor is integrated into the exterior surface of the device. The method can include capturing an image or impression from the anatomical region of interest applied to the sensor and guiding placement of the anatomical region of interest in relation to the sensor using the image or impression. The method can include constructing two or more oscillograms for the user from the measured pressure and the measured blood volume oscillations, wherein each oscillogram in the two or more oscillograms is constructed from measurements taken at different placements of the anatomy of interest in relation to the sensor. The method can include determining a particular placement for the anatomy of the user based on at least one attribute of the two or more oscillograms, and guiding the user to place the anatomy of interest at the particular placement. If desired, the sensor can include a photoplethysmography (PPG) sensor and a pressure sensor.

The disclosure also provides a device for measuring blood pressure including detection, processing and display circuitry. The device includes first circuit configured and arranged to guide a user regarding placement of an anatomical region of interest in relation to a sensor, a second circuit configured and arranged to measure, by the sensor, a measured pressure applied to the sensor, a third circuit configured and arranged to measure, by the sensor, measured blood volume oscillations in the anatomical region of interest while pressure is being applied to the sensor by the anatomical region of interest, a fourth circuit configured and arranged to estimate, by the processing circuitry of the device, a blood pressure value for the user from the measured pressure and the measured blood volume oscillations, and a fifth circuit configured and arranged to present the blood pressure value.

In some implementations, the first circuit can be configured and arranged to place a visual indicator on an exterior surface of the device, wherein the visual indicator is arranged in relation to the sensor and the sensor is integrated into the exterior surface of the device. The first circuit can be configured and arranged to capture an image or impression from the anatomical region of interest applied to the sensor and to guide placement of the anatomical region of interest in relation to the sensor using the image or impression. The third circuit can be configured and arranged to construct two or more oscillograms for the user from the measured pressure and the measured blood volume oscillations, where each oscillogram in the two or more oscillograms is constructed from measurements taken at different placements of the anatomy of interest in relation to the sensor.

If desired, the device can be further configured to determine a particular placement for the anatomy of the user based on at least one attribute of the two or more oscillograms, and to guide the user to place the anatomy of interest at the particular placement. The sensor can include a photoplethysmography (PPG) sensor and a pressure sensor. The PPG sensor can be a reflectance-mode PPG sensor array with a surface having a surface area of about 5 mm² to about 500 mm². The PPG sensor can include at least one photodetector and at least one light emitting diode (LED). The PPG sensor can be an array of PPG sensors including a plurality of PPG sensors and further wherein the device is configured and arranged to choose the sensor that yields a largest amplitude oscillogram. The pressure sensor can be a thin-filmed transducer. The pressure sensor can include a plurality of pressure sensors, and further wherein each pressure sensor is configured and arranged to measure a corresponding applied pressure. For each of the plurality of pressure sensors, the device can be configured to measure the corresponding pressure.

The disclosure also provides implementations of a processor-readable tangible non-transient medium storing a computer program for operating an arrangement of processing circuitry including a device for measuring blood pressure communicatively coupled to at least one processing circuit. The computer program can include instructions configured to guide a user regarding placement of an anatomical region of interest in relation to a sensor, instructions configured to measure, by the sensor, a measured pressure applied to the sensor, instructions configured to measure, by the sensor, measured blood volume oscillations in the anatomical region of interest while pressure is being applied to the sensor by the anatomical region of interest, instructions configured to estimate, by the processing circuitry of the device, a blood pressure value for the user from the measured pressure and the measured blood volume oscillations, and instructions configured to present the blood pressure value.

The processor-readable tangible non-transient medium can further include instructions configured to place a visual indicator on an exterior surface of the device, wherein the visual indicator is arranged in relation to the sensor and the sensor is integrated into the exterior surface of the device. The computer program can further include instructions configured to capture an image or impression from the anatomical region of interest applied to the sensor and to guide placement of the anatomical region of interest in relation to the sensor using the image or impression. The computer program can further include instructions configured to construct two or more oscillograms for the user from the measured pressure and the measured blood volume oscillations, wherein each oscillogram in the two or more oscillograms is constructed from measurements taken at different placements of the anatomy of interest in relation to the sensor.

If desired, the computer program can further include instructions configured to determine a particular placement for the anatomy of the user based on at least one attribute of the two or more oscillograms, and to guide the user to place the anatomy of interest at the particular placement. If desired, the device can includes a PPG sensor having an array of PPG sensors and the computer program can further include instructions configured to choose the sensor that yields a largest amplitude oscillogram. The device can further include a plurality of pressure sensors, and the computer program can further include instructions configured to measure a corresponding applied pressure from each pressure sensor.

It is to be understood that both the foregoing general description and the following detailed description are exemplary and are intended to provide further explanation of the embodiments disclosed herein.

The accompanying drawings, which are incorporated in and constitute part of this specification, are included to illustrate and provide a further understanding of the method and system of the disclosure. Together with the description, the drawings serve to explain the principles of the disclosed embodiments.

### DESCRIPTION OF THE DRAWINGS

The drawings described herein are for illustrative purposes only of selected embodiments and not all possible implementations, and are not intended to limit the scope of the present disclosure.

FIG. 1 is a diagram depicting an example embodiment of a mobile device that embodies cuff-less BP measurements.

FIGS. 2A-2C are diagrams depicting an embodiment of the cuff-less blood measurement system on the mobile device.

FIG. 3 is a diagram depicting an example embodiment of a cuff-less system in a mobile device that obtains BP measurements.

FIG. 4 is a flowchart depicting an example embodiment of a cuff-less system in a mobile device that obtains BP measurements.

FIG. 5 is a diagram depicting an example embodiment of the sensor.

FIGS. 6A and 6B are an example embodiment of how BP is estimated using a Standard Fixed-Ratio Algorithm.

FIGS. 7A and 7B are diagrams depicting an example embodiment of how BP is estimated using a Patient-Specific Algorithm.

FIG. 8 is a diagram depicting a prototype system that was created to test the feasibility of the oscillometric finger pressing paradigm.

FIGS. 9A and 9B are graphs depicting the pressure applied to the sensor and the target pressure displayed when pressure is applied to the sensor as well as the oscillogram that is generated to determine BP.

FIG. 10 is a diagram depicting an index finger that exerts the pressure applied to the sensor of the prototype system.

FIGS. 11A-11C are diagrams depicting results of testing a prototype system.

FIG. 12 is a diagram depicting an example embodiment of cuff-less BP measurement devices.

FIG. 13 is a diagram depicting an example PPG sensor on the sensor.

FIGS. 14A and 14B are diagrams depicting example embodiments of sensors coupled to an encasing of mobile devices.

FIG. 15 is a diagram depicting another embodiment of the cuff-less BP measurement system on the mobile device.

FIG. 16 is a diagram depicting another embodiment of the cuff-less BP measurement system

FIGS. 17A-17C are diagrams depicting an embodiment of the cuff-less BP measurement system with finger placement indicators.

FIGS. 18A-18D are diagrams depicting an example position detection system included in the cuff-less BP measurement system.

FIG. 19 is a flowchart depicting the example position detection system included in the cuff-less BP measurement system.

FIGS. 20 and 21 illustrate aspects of a computer system in accordance with the present disclosure for storing and analyzing blood pressure and other health-related or medical data.

Corresponding reference numerals indicate corresponding parts throughout the several views of the drawings.

### DETAILED DESCRIPTION

Example embodiments will now be described more fully with reference to the accompanying drawings.

The present disclosure relates to reliable methods, systems and machine readable programs for cuff-less BP monitoring via the oscillometric principle. In conventional oscillometry, an inflatable cuff serves as both an actuator to vary the external pressure of an artery and a sensor to measure this pressure and the resulting variable-amplitude blood volume oscillations in the artery. BP is then estimated from the oscillation amplitudes as a function of the applied pressure (again, the “oscillogram”). The present disclosure is to extend the oscillometric principle for cuff-less monitoring of BP using a smartphone, another mobile device (e.g., PDAs, laptops, tablets, and wearables), and/or possibly an encasing of a mobile device, and using it to measure blood pressure at a variety of locations of the anatomy.

In some implementations, the user serves as the actuator by pressing a desired anatomical location against the mobile device, or against a mobile device accessory, or an electronic sensor otherwise connected to a computing device, preferably at heart level to steadily increase the external pressure of the underlying artery. Such user actuation may afford external pressure application similar to a cuff in that the artery of interest will be pressed against supporting bone or other anatomical structure. The mobile device preferably provides visual and/or auditory or tactile (e.g, vibratory) guidance for proper actuation, measures the applied pressure and blood volume oscillations, and estimates BP from the oscillogram. Embodiments in accordance with the disclosure can be implemented with a photoplethysmography (PPG) sensor, which measures pulsatile blood volume and a pressure sensor embedded in a smartphone encasing or within the phone itself, or alternatively may be performed with a peripheral I/O computing device. By having the user serve as the actuator in these embodiments, the requisite hardware is automatically miniaturized and greatly simplified. Note that the mobile device may also warn users of high BP, securely transmit the measured BP to caregivers, and send text reminders to patients with uncontrolled BP to take their medications. In this way, a complete hypertension management system can thus be available to many.

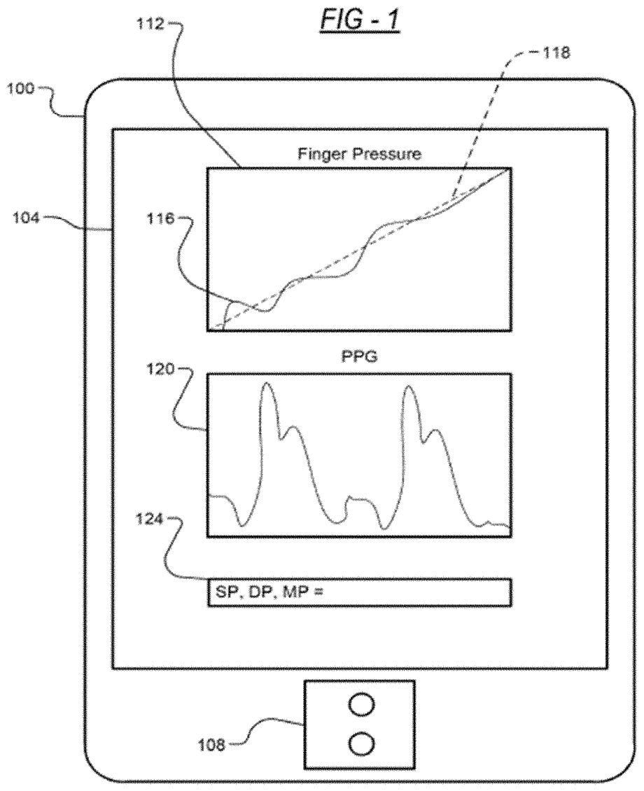

FIG. 1 is a diagram depicting an example embodiment of a cuff-less BP measurement system. The mobile device 100 includes processing circuitry (e.g., processors and the like) operably coupled to a display 104 including display circuity operably coupled to the processing circuitry and a sensor 108 including sensor circuitry. The display 104 may present graphics that depict, in a single graph 112, a pressure applied 116 by an anatomical location of interest where an artery is located to the sensor 108 over a target pressure 118 to be exerted, blood volume oscillations 120 from the sensor 108, as well as the SP, DP and MP of the user as indicated at 124 of the display. In an example embodiment, the user presses their anatomical location of interest against the sensor 108 to steadily increase the external pressure of the underlying artery, while the sensor 108, which includes a photoplethysmography (PPG) sensor and a pressure sensor, detects and measures the blood volume oscillations and the pressure applied 116 to the sensor 108. Using the measured blood volume oscillations 120 and the pressure applied 116 to the sensor 108, the oscillogram (again, the oscillations amplitudes as a function of applied pressure) is generated. From the oscillogram, the SP, DP, and MP of the user are determined and presented, for example using a display of the device. In addition, the pressure applied 116 to the sensor 108 is displayed in the graph 112, and the blood volume oscillations 120 are displayed. The mobile device 100 includes but is not limited to mobile phones, PDAs, laptops, tablets, and wearable devices (e.g., watches).

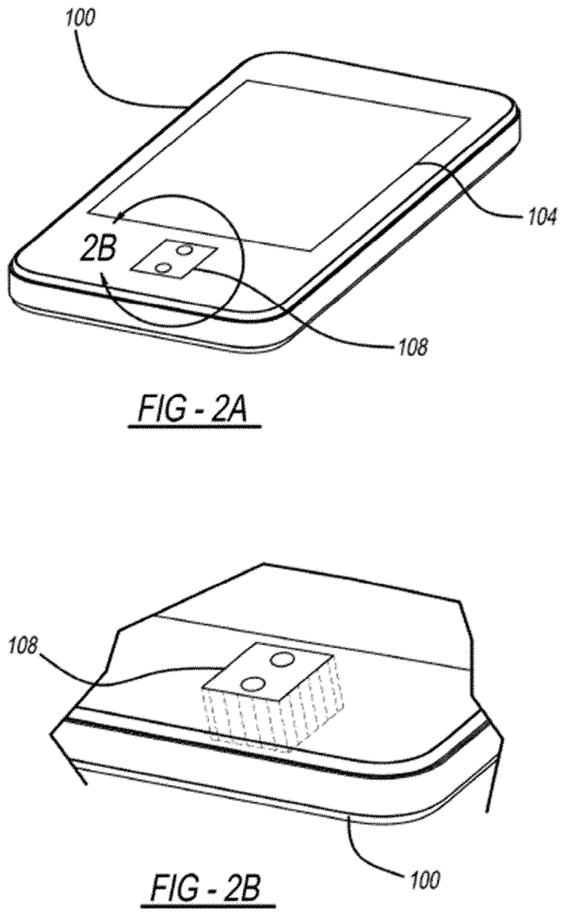

FIGS. 2A-2C are diagrams further depicting the embodiment of the cuff-less BP measurement system on the mobile device 100. The user serves as the actuator by pressing their anatomical location of interest against the mobile device 100, on the sensor 108, held at heart level to steadily increase the external pressure of the underlying artery. Manual user actuation can afford external pressure application similar to a cuff in that the artery will be pressed against supporting bone or other supporting tissue.

The mobile device 100 provides visual guidance on the display 104 for proper actuation. That is, having the graph 112 of the pressure applied 116 to the sensor 108 on the same graph 112 as the target pressure 118 provides the user with visual feedback as to how much pressure to exert. The sensor 108 also measures blood volume oscillations 120 to generate an oscillogram 128, and BP is estimated from the oscillogram 128. The pressure applied 116 to the sensor 108 is graphed over a target pressure 118 to guide the user on the need to apply increased pressure and when to apply increased pressure. Graphing the pressure applied 116 to the sensor 108 in real time over the target pressure 118 allows the user to attempt to trace the target pressure 118. By having the user serve as the actuator, the requisite hardware is automatically miniaturized and greatly simplified. The SP, the DP, and the MP can be calculated from the oscillogram 128.

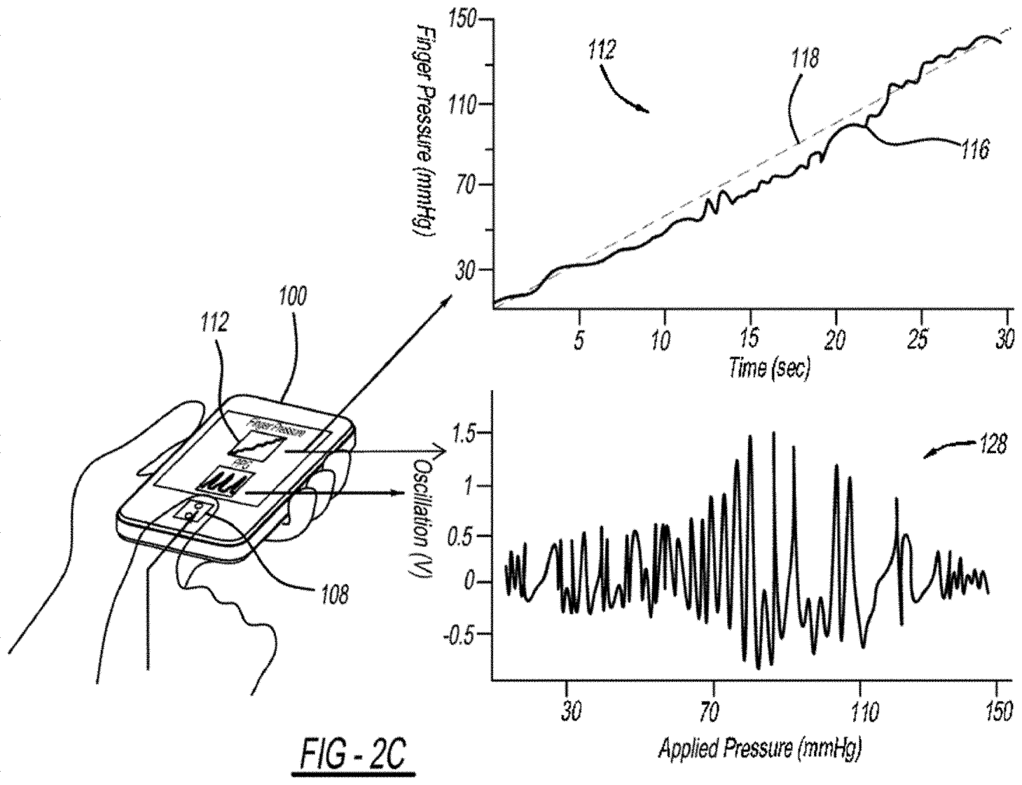

FIG. 3 is a diagram depicting an example embodiment of a cuff-less system that obtains BP measurements that may be implemented in the mobile device 100. The system includes a display 104, a sensor 108, a computer processor 300 and a data store 304 (e.g., a non-transitory storage medium). The system further includes an oscillogram generator 308, a BP estimator 312, and a pressure guide 316, which are implemented, for example, as computer-executable instructions residing in the data store 304 and executed by the computer processor 300.

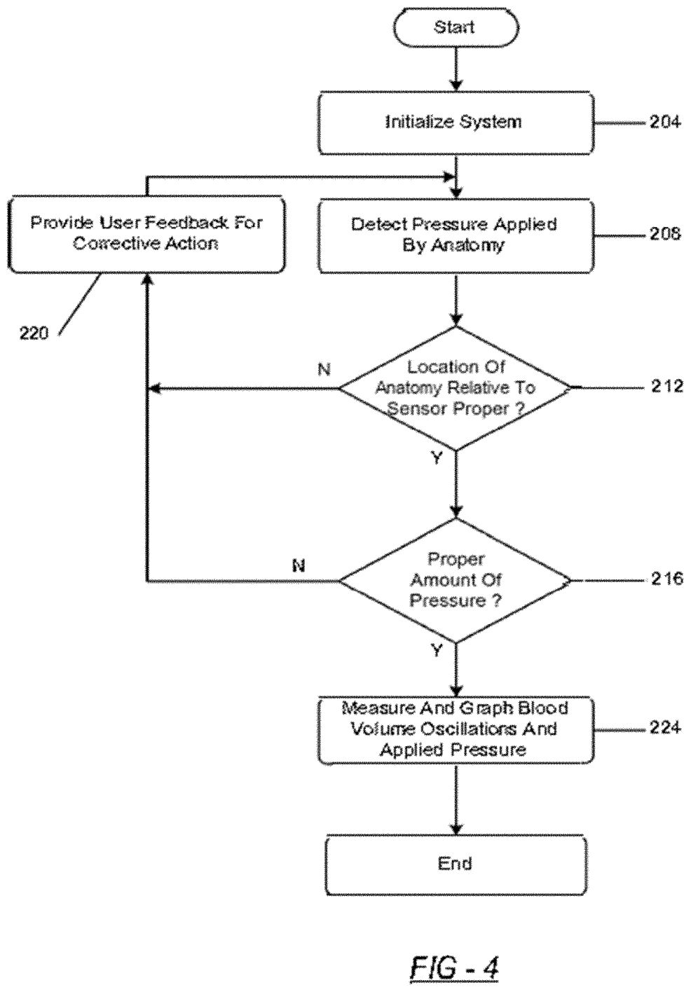

The sensor 108 is operably coupled to the computer processor 300 of the mobile device 100. The sensor 108 can be a sensor array including a plurality of sensors and supporting circuitry including a PPG sensor 320, a pressure sensor 324, and possibly a temperature sensor 326. The temperature sensor 326 is optional, and the BP measurements may be obtained without it. The PPG sensor 320 of the sensor 108 is operably coupled to the oscillogram generator 308, and the pressure sensor 324 is operably coupled to the oscillogram generator 308 and the pressure guide 30. The sensor 108 is configured to communicate the measured values of the PPG sensor 320 and the pressure sensor 324 to the computer processor 300 of the mobile device 100. An example embodiment of the sensor 108 is further described below in FIG. 5.

The oscillogram generator 308 is configured to generate an oscillogram based on input from the PPG sensor 320 and input from the pressure sensor 324. In an example embodiment, an oscillogram is constructed by first taking a maximum value and a minimum value of each beat of the blood volume waveform that is detected and measured by the PPG sensor 320. The maximum value and minimum value of each beat, as a function of the pressure applied 116 to the sensor 108 (obtained by the pressure sensor 324), are then median filtered to attenuate respiratory and heart rate variability. Finally, the maximum value and minimum value of each beat are linearly interpolated, and the difference between the two envelopes is taken as the oscillogram 128. Although not limited thereto, the oscillogram generator 308 may generate an oscillogram 128 using other known algorithms as would be understood by one having skill in the art.

In extending this algorithm of generating the oscillogram 128 using manual pressing instead of a cuff, issues of detecting beats in the presence of artifact and connecting the extrema of valid beats, which can be separated by a wide range of the pressure applied 116 to the sensor 108, may be present. To overcome these issues, algorithms that first identify artifact in the blood volume waveform that exploit the anticipated blood volume shape and then detect the maxima and minima of the artifact-free beats can be implemented into the system. Advanced filtering and splining algorithms, as well as parametric model (Gaussian functions) fitting, which may be more robust, can be used to connect the extrema of the clean beats.

To assess the validity of the oscillogram 128, various features such as the number of artifact-free beats, the applied pressure range over which these beats extend, and the shape, width, and degree of symmetry of the oscillogram 128 may be analyzed to determine the validity of the oscillogram 128. An algorithm such as linear discriminant analysis may be implemented to distinguish between valid and invalid oscillograms based on these features.

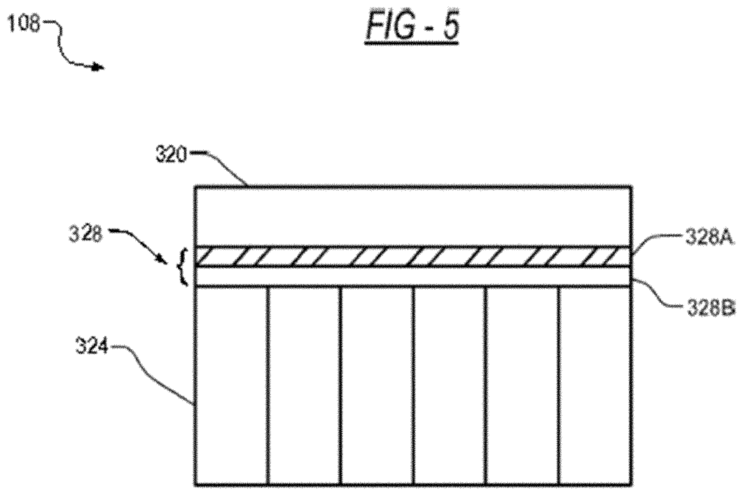

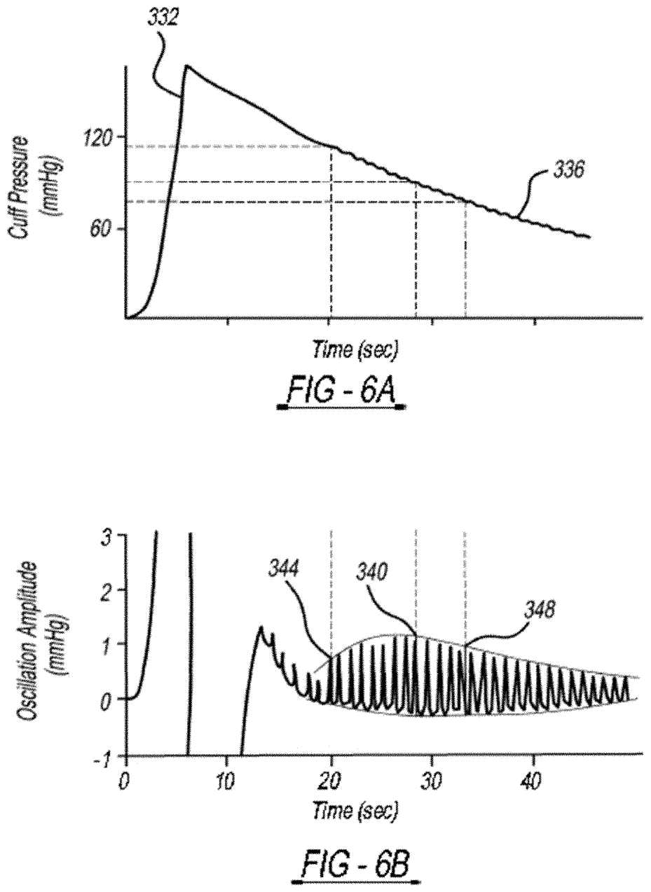

The BP estimator 312 is configured to determine the BP based on the oscillogram 128 generated by the oscillogram generator 308. Subsequently, the BP estimator 312 presents the BP value on the display 104. Example algorithms that may be used in estimating BP are the Standard Fixed-Ratio Algorithm, the Fixed-Slope Algorithm, a Patient-Specific Algorithm, and other variations of these algorithms. These algorithms may also be combined in various manners to estimate BP. In addition, an age-dependent scaling algorithm of SP at the anatomical location of interest may be used to estimate brachial SP, since the ratio of SP to brachial SP may decrease with age. Brachial BP may also be determined from model-based transfer functions. While using the model-based transfer functions would require an input of the BP waveform, the BP waveform may be obtained using a Patient-Specific Algorithm. Some example embodiments of algorithms that may be used in estimating the BP are further described below in relation to FIGS. 6A-7B.

The pressure guide 316 may optionally be interfaced with the pressure sensor 324. In some embodiments, the pressure guide 316 scales the pressure applied to the pressure sensor 324 to a measure of pressure applied 116 to the sensor 108 exerted on the PPG sensor 320. The pressure guide 316 is also configured to present the estimated magnitude of the pressure applied to the sensor 108 on the display 104. By displaying the amount of pressure applied to the sensor 108 on the display 104, the pressure guide 316 also provides the user with real time feedback regarding the amount of pressure applied to the sensor 108 and the location of the anatomical location of interest relative to the sensor 108, as described further below. Thus, the user can take corrective action based on the real-time feedback so that the target pressure 118 can be applied to the sensor 108 for a predetermined period of time. The pressure guide 316 also receives feedback from the temperature sensor 326. The temperature sensor 326 measures a temperature of the anatomical location of interest applying pressure 116 to the sensor unit 108. Under the circumstance that the temperature of the anatomical location of interest is too low or possibly too high, the display provides feedback informing the user that the anatomical location of interest temperature is outside of an acceptable range, which can affect the results of the BP measurement system.

In an example embodiment, the oscillogram generator 308, BP estimator 312, and the pressure guide 316 may be implemented on the mobile device 100 as an application. The application can be used to guide the manual actuation, inform the user of any adjustments required in the pressure applied 116 to the sensor 108 or placement of the anatomical location of interest, graph applied pressure and possibly the blood volume oscillations 120, and display the graphs along with the SP, DP, and MP/BP. The application uses the display 104 and processor of the mobile device. For example, the application provides visual feedback to guide the actuation by graphing the pressure applied 116 to the sensor 108 over the target pressure 118. That is, the target pressure 118 may be a linear target rise or a pressure in step increments, which may yield more artifact-robust oscillograms over certain time interval (e.g., at least 15 sec). The pressure applied 116 to the sensor 108 is superimposed as it is being recorded in real-time.

Alternatively, a display of the pressure applied 116 to the sensor 108 as it evolves in real-time within a plotting window that tells the user to raise the pressure steadily to a high level (e.g., 150 mmHg) over fixed time interval, but not in any preset way, may be used. A third option is to guide the actuation through a video game that requires the user press at various pressures to accomplish the goals of the game. In addition or in another embodiment, audio feedback can be used to guide the actuation.

Additionally, after the BP has been computed, the application may determine whether the BP is within an acceptable range. If the BP falls outside of the acceptable range, the application may instruct the user to repeat the BP measurement in order to ensure accuracy. The application then displays the computed BP and other physiologic variables, if available, or asks the user to repeat the procedure in the event of an unsuccessful actuation. The application can also ask the user to repeat the procedure even when the actuation is deemed successful. For example, the application can average two similar BP measurements or the two closest BP measurements out of three total measurements to reduce variability. The application may also alert the user if the BP is too high or too low, securely transmit the measurements to the cloud as well as the physician, and send text reminders to users with repeatedly high BP measurements to take their medications. The application may further allow the user to view their history of BP measurements over time and integrate with other health and lifestyle applications on the mobile device 100 such as those that track eating habits.

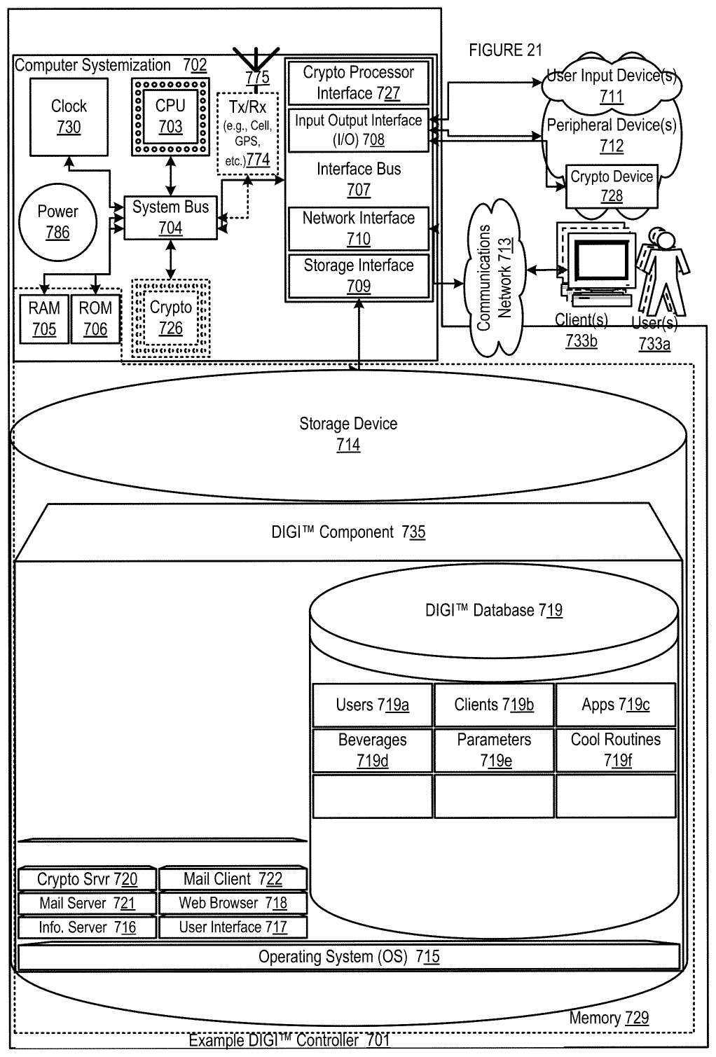

The data store 304 is interfaced with the BP estimator 312 and the pressure guide 316. The data store 304 is configured to store BP values (MP, DP, and SP) that have been determined by the BP estimator 312. Pressure values from the pressure sensor 324 and PPG values from the PPG sensor 320 may also be stored in the data store 304. This may be useful for a user who is interested in tracking and analyzing BP values over a period of time to determine whether lifestyle changes, dietary changes, and/or exercise routines are improving her BP and overall cardiovascular health. The data store 304 may also be configured to provide the computer processor 300 of the mobile device 100 with processor readable instructions for the oscillogram generator 308, the pressure guide 316, and the BP estimator 312. As an example, the data store 304 may provide the computer processor 300 of the mobile device 100 with executable instructions that allow it to generate an oscillogram 128 from the blood volume oscillations detected and measured by the PPG sensor 320 and the pressure applied 116 to the sensor 108 detected and measured in the pressure sensor 324. The data store 304 may also provide the computer processor 300 of the mobile device 100 with executable instructions to estimate BP based on the oscillogram 128 generated by the oscillogram generator 308 and an algorithm that estimates BP based on certain parameters of the oscillogram 128. All values can be uploaded to and monitored in a data management system as illustrated in FIGS. 20-21.

The display 104 may provide the user with real-time feedback regarding the pressure applied 116 to the sensor 108 and the location of the anatomical location of interest relative to the sensor 108. As an example, the system may provide the user visual feedback when the pressure applied 116 to the sensor 108 is below a target pressure 118. As another example, the system may provide the user visual feedback when the location of the anatomical location of interest is not at a predefined optimal location that allows for optimal oscillogram measurements. The predefined location may be determined by an initialization protocol, which occurs when the actuation is attempted over a range of locations on the sensor, and the location that yields the largest oscillogram amplitude is selected as the predefined optimal anatomical location.

To guide the user in increasing the pressure applied 116 to the sensor 108, the target pressure 118 may have a trajectory of a linear rise, a step increment, or a combination of a step increment and a linear rise shown on the display 104. In other embodiments, the target pressure 118 may not be displayed. For example, the display 104 can include the desired start and end pressures with the time interval to reach the end pressure.

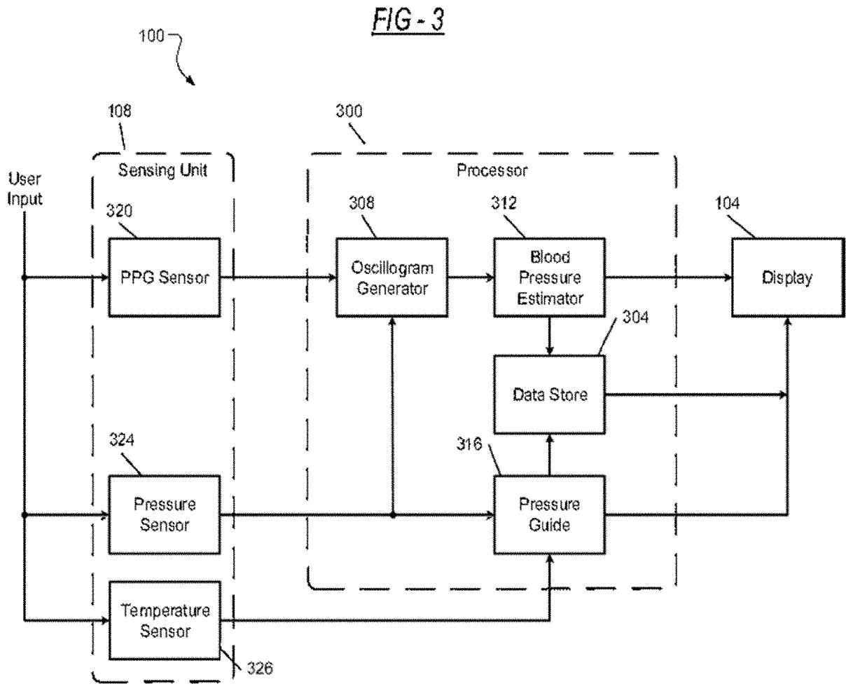

FIG. 4 is a flowchart depicting an example embodiment of a cuff-less system that obtains BP measurements. The flowchart depicts an example of a determination of proper anatomical positioning and proper pressure applied 116 as may be implemented by the pressure guide 316 of FIG. 3. First, the system is initialized at 204. During this stage, the mobile device may initialize the display and the sensor (i.e., opening an application on the mobile device 100 and/or turning on the sensor 108) to measure the pressure applied 116 to the sensor 108, the blood volume oscillations 120 with the sensor 108, the oscillogram 128, and the SP, DP, and MP/BP and display the measurements once the user begins to apply pressure to the sensor 108. The mobile device 100 may also, using information loaded in a data storage unit, load the user's predefined optimal anatomical placement location data. The user data may be accessed by the initialization protocol by inserting a username or ID and a password to load the user's predefined optimal anatomical placement location data.

Second, the system begins to detect the pressure applied 116 to the sensor 108 at 208. Next, the system provides the user with real-time feedback. At 212 the system then determines whether the location of the anatomy relative to the sensor is proper, wherein the proper location is the predefined optimal location for the anatomy with respect to the sensor. If so, then at 216 the system determines whether the user is applying the proper amount of pressure, wherein the proper amount of pressure is the target pressure 118. If so, the system proceeds to the next step.

If the location of the anatomy of interest relative to the sensor 108 is not proper at 212, or if the amount of pressure is not proper at 216, the system, at 220, provides corrective feedback so that the user can either correct the amount of pressure applied to the sensor 108 or adjust her anatomical positioning relative to the sensor 108. As an example, the feedback may instruct the user to either increase or decrease the amount of pressure applied so that the user can apply the target pressure 118. As another example, the feedback may instruct the user to adjust the positioning of her anatomy of interest so that the positioning of the anatomy is at the predefined optimal anatomical location. Control then proceeds to 208. The feedback may be visual, audio-based, or a combination of visual and audio-based feedback.

Once the target pressure 118 is met and proper anatomical positioning is achieved, the sensor 108 measures and graphs the blood volume oscillations and the pressure applied 116 to the sensor 108 at 224. The system then displays the BP to the user. The system determines the SP and DP based on the blood volume oscillations and the pressure applied 116 to the sensor 108. Using the SP and DP, the system estimates the MP/BP from the blood volume oscillations and the pressure applied 116 to the sensor 108 using various BP estimation algorithms. Depending on the determination method, the MP/BP may be determined first followed by the SP and DP. In any case, the mobile device 100 subsequently displays the MP/BP.

FIG. 5 is a diagram depicting an example embodiment of the sensor 108. The sensor 108 includes a PPG sensor 320 and a pressure sensor 324, wherein the PPG sensor 320 and the pressure sensor 324 are coupled to each other by an interface unit 328. The PPG sensor 320 and the pressure sensor 324 may be coupled to the computer processor 300 of the mobile device 100, wherein the computer processor 300 of the mobile device 100 may subsequently determine the BP from the detected values of the PPG sensor 320 and the pressure sensor 324. The sensor 320 surface may be flat or concave to facilitate anatomical positioning on the sensor 108.

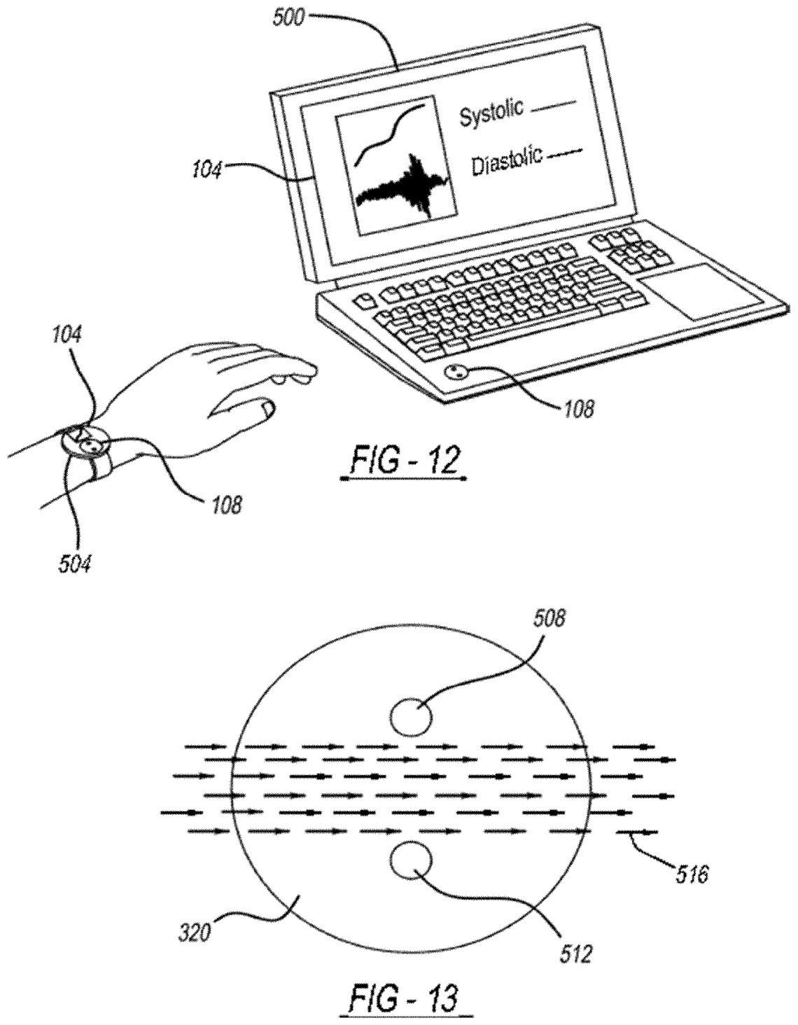

In an example embodiment, the PPG sensor 320 is an infrared, reflectance-mode PPG sensor that measures blood volume oscillations from the arteries beneath the skin. The PPG sensor 320 may be configured in a way such that the blood volume oscillations of a particular artery of interest can be accurately and efficiently recorded. An LED and a photodetector (referenced and discussed in FIG. 13) of the PPG sensor 320 may be positioned perpendicular to the artery of interest, wherein the LED and the photodetector are separated by a fixed distance. The fixed distance may be chosen such that the blood volume oscillation amplitudes detected and measured by the PPG sensor 320 are maximized.

In an example embodiment, the pressure sensor 324 is a thin-filmed capacitive transducer. The transducer outputs the pressure applied 116 to the sensor 108 in the normal direction. The pressure sensor 324 may be configured to output a pressure between the range of O to 250 mmHg at an output resolution of less than 0.1 mmHg. Other pressure sensors that are configured to output a force when the pressure applied 116 to the sensor 108 may also be used instead of the thin-filmed capacitive pressure sensor 324 described in this embodiment.

In an example embodiment, the interface unit 328 is a thin, rigid structure 328A adhesively coupled to a foam material 328B. The rigid structure of the interface unit 328A is coupled to the PPG sensor 320, while the foam material of the interface unit 328B is coupled to the pressure sensor 324. This interface unit 328 allows for the force applied to the PPG sensor 320 to be distributed uniformly to the pressure sensor 324.

Alternatively, a silicone layer or similar material may be used in place of the foam material 328B. Other materials that may be used in place of the foam material 328B are materials that are configured to distribute an applied force evenly over its respective area and acts as a mechanical low-pass filter to mitigate the impact of any spurious pressing of the anatomy of interest against the sensor.

A surface of the sensor 108 that receives the pressure applied 116 to the sensor 108 from the user should have an area that is optimized in order to allow for reliable BP estimation. For example, in certain embodiments, if the area of the surface is too large, then substantial force will be needed to achieve the target pressure 118. If the area of the surface is too small, then modest variations in pressure applied 116 to the sensor 108 will induce substantial pressure changes. The area of the surface of the sensor 108 that receives the applied force from the subject should therefore be optimized to allow for the sensor 108 to measure and detect the pressure applied 116 to the sensor 108 that achieves an optimal balance between these two considerations.

FIGS. 6A and 6B are an example embodiment of how BP is estimated using a Standard Fixed-Ratio Algorithm. As described above, the oscillogram generator 308 receives data from the PPG sensor 320 and the pressure sensor 324 (e.g., the pressure applied 116 as depicted in FIG. 6A at 332 and 336) to generate an oscillogram.

FIG. 6A is a diagram depicting the pressure applied 116 to an artery. A cuff is placed around a patient's arm and inflated. The cuff is inflated while secured around the patient's arm, shown in FIG. 6A where the cuff pressure is increasing rapidly 332. Once the cuff reaches a target external pressure to the artery, the cuff slowly deflates 336. While deflating 336, the cuff measures a blood volume value and external pressure and constructs an oscillogram from the resulting variable amplitude blood volume oscillations shown in FIG. 6B. The mean BP (MP) 340 is estimated as the pressure at which the oscillogram is at a maximum amplitude 340 (AM)—Then, an amplitude of the SP (As) 344 and an amplitude of the (Ao) DP 348 are estimated as the pressure at which the oscillogram is a fixed, population based average ratio of its maximal value (As/AM and A0/AM)—The ratios in this embodiment are fixed such that As/AM and A0/AM are equal to 0.55 and 0.85, respectively. While this method depicts measuring BP using the cuff, this algorithm can be applied in a cuff-less system as well, wherein pressure against the anatomy of interest is used instead of cuff pressure.

However, since the Standard Fixed-Ratio Algorithm is population based, the algorithm may be less effective in accurately determining BP levels for those individuals who have BP not within a normal BP range. The BP estimation errors of the Standard Fixed-Ratio Algorithm may be significant and may be impacted by the width of the arterial compliance curve, which is the derivative of the blood volume transmural pressure relationship with respect to transmural pressure. The accuracy of the Standard Fixed-Ratio Algorithm may also be affected by those who have a high pulse pressure (i.e., the difference between the SP and DP) due to artery stiffening, a common condition that occurs with aging and disease.

FIGS. 7A-7B are diagrams depicting an example embodiment of how BP is estimated using a Patient-Specific Algorithm. The oscillograms depicted at 368, and 384 are produced by the oscillogram generator 308 described in FIGS. 3. The Patient-Specific Algorithm represents the oscillogram 128 with a physiologic model and then estimates the patient-specific model parameters, which include BP levels and reflect the width and other features of the arterial compliance curve by optimally fitting the model to the oscillogram 128. Thus, accuracy can be maintained over a wide BP range. Furthermore, by employing a physiologic model, the method can be more robust to deviations in the oscillogram 128 caused by respiration and heart rate variability and thus be more repeatable and reliable. While this method depicts measuring BP using a cuff, this algorithm can be applied in a cuff-less system as well, wherein pressure against the anatomy of interest is used instead of cuff pressure.

In FIG. 7A, air volume versus cuff pressure is shown for two different types of cuffs: a dura cuff 352 and a bladder cuff 356. From the nearly linear relationship displayed in FIG. 7A, a cuff compliance or scale factor k 360 is determined.

The first step of estimating BP using the Patient-Specific Algorithm is to represent the cuff pressure oscillation amplitude versus the cuff pressure function (i.e., the oscillogram) with a parametric model of the nonlinear Brachial artery blood volume-transmural pressure relationship. This representation is demonstrated in the following equation 1:

$$\overbrace{\text{?}}_{Red Envelope Difference} = {\overbrace{e}}_{k - d} \underbrace{{\{ 1 + [ {b}^{- 1} ( ( SP - {P}_{c} ( t ) - a ) + {b ( \frac{c - 1}{c + 1} )}^{1 / c} ) ] \text{?} \}}^{- 1}}_{Nonlinear relationship at systoic} \cdot e \underbrace{{\{ 1 + [ {b}^{- 1} ( ( DP - {P}_{c} ( t ) - a ) + {b ( \frac{c - 1}{c + 1} )}^{1 / c} ) ] \text{?} \}}^{- 1}}_{Nonlinear relationship at diastolic}$$

$$\text{?} \text{indicates text missing or illegible when filed}$$

The unknown parameters (a, b, c, and e) represent the SP, DP, and Brachial artery mechanics. In terms of the Brachial artery compliance curve (i.e., the derivative of the nonlinear relationship with respect to transmural pressure), parameter a represents the transmural pressure at which the curve is a maximum; parameters b and c denote the width of the curve and the extent of the asymmetry about its maximum; and parameter e indicates the amplitude of the curve. The parameter e is determined by the reciprocal of the cuff compliance, which is represented by scale factor k 360. The scale factor k 360 is assumed to be constant as justified by experimental data. A blood volume 372 is determined based on (i) the nearly linear relationship of the cuff pressure and air volume 356, (ii) blood volume oscillations 364, and (ii) cuff pressure oscillations 368. The envelope differences of the blood volume 372 are equal to within scale factor k 360.

The second step of estimating BP using the Patient-Specific Algorithm is to estimate the model parameters including the SP and DP by fitting the model to the oscillogram. The model parameters are estimated using the following equation 2:

$${min}_{\{ a , b , c , e , … , SP , DP \}} \text{?} [ \text{?} {\{ 1 + [ {b}^{- 1} ( ( SP - {P}_{c} ( t ) - a ) + {b ( \frac{c - 1}{c + 1} )}^{1 / c} ) ] \text{?} \}}^{- 1} - \text{?} {\{ 1 + [ {b}^{- 1} ( ( DP - {P}_{c} ( t ) - a ) + {b ( \frac{c - 1}{c + 1} )}^{1 / c} ) ] \text{?} \}}^{- 1} \\ \text{?} \text{indicates text missing or illegible when filed}$$

The first step and the second step yield estimates for SP and DP as well as parameters a, b, c, and e, which characterize the underlying model of the nonlinear

Brachial artery blood-volume transmural pressure relationship. The third and fourth step use the parameter estimates to ultimately yield an estimate for the entire Brachial BP waveform (Pb(t)) and MP, as described next.

The third step of estimating BP using the Patient-Specific Algorithm, which is shown in FIG. 7B, is to construct the blood volume waveform to within the scale factor k 360 using the parameter estimates (i.e., a, b, c, and e) and cuff pressure oscillations. Positive cuff pressure oscillations 376 are calculated by subtracting a lower cuff pressure oscillation envelope 380 from cuff pressure oscillations 384. Then, the blood volume waveform to within scale factor k 388 is calculated by adding a modeled arterial blood volume-transmural pressure relationship at diastole to within scale factor k 392.

The fourth step of estimating BP using the Patient-Specific Algorithm is to construct the BP waveform using the blood volume waveform 388 via root finding. From the BP waveform, MP is computed as the time average of the derived waveform. The following equation 3 illustrates how the BP waveform and the MP are derived:

Further details regarding the Patient-Specific Algorithm may be found in U.S. Provisional Application No. 62/217,331 filed Sep. 11, 2015 incorporated by reference in its entirety herein.

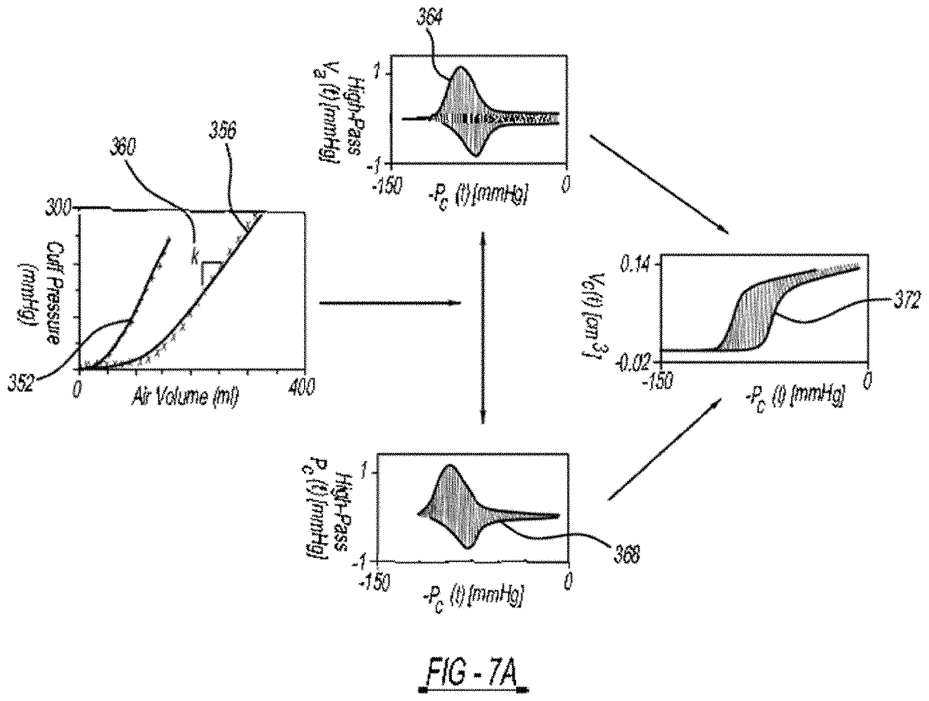

The oscillometric principles of an inflatable cuff as described in FIGS. 6A-7B are applied to cuff-less BP monitoring as shown in a prototype system 394. FIG. 8 is a diagram depicting the prototype system 394 that was created to test the feasibility of the oscillometric finger pressing paradigm, as an example.

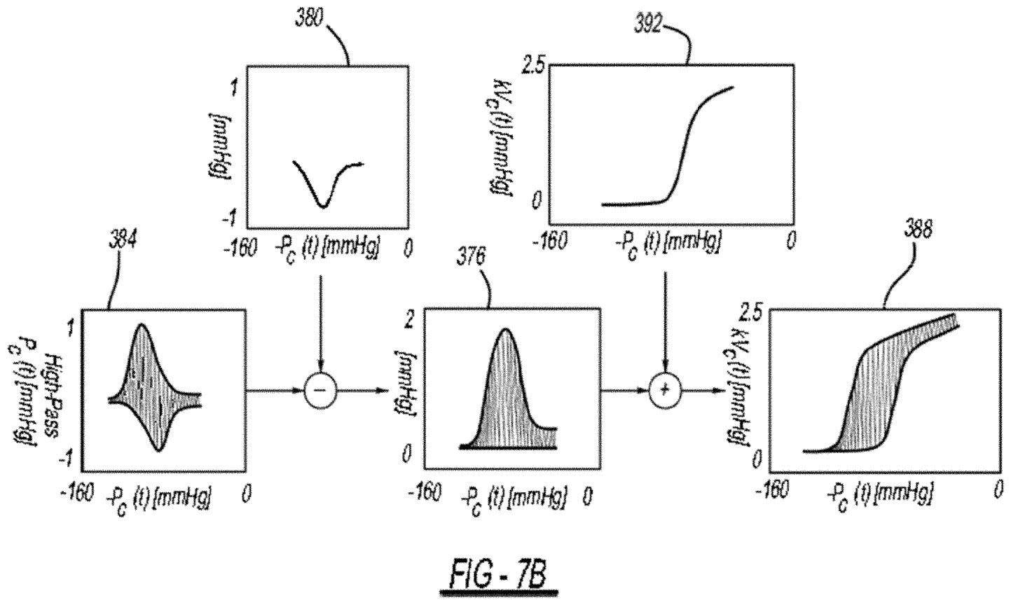

The prototype system 394 consists of a simple sensor unit interfaced to a computer, which provides a visual display, as shown in FIGS. 9A and 98, and runs standard algorithms to estimate BP.

The sensor includes the PPG sensor 320 and pressure transducers as the pressure sensor 324 housed in a plastic enclosure. The PPG sensor 320 is an LED and photodetector operating in reflectance-mode and at an infrared wavelength (940 nm) to penetrate an artery beneath the skin. The PPG sensor 320 surface, which constitutes the pressing area, in this example, with respect to an arterial measurement on a finger, is a 10 mm diameter circle. Other shapes and sizes are possible, such as ellipses, rectangles, squares, serpentine shapes, linear shapes, and the like, having areas ranging from about 5 mm² to about 500 mm², and in any desired increment therebetween of 1 mm².

The pressure sensor 324 (DigiTacts Sensors, Pressure Profiling Systems, USA) is a thin-filmed, 16×3 array of capacitive transducer elements (5 mm length squares). Each element outputs the pressure exerted on it in the normal direction and has specifications that are congruent with BP measurement (e.g., resolution and range are <1 mmHg and >250 mmHg). The PPG sensor 320 is on top of the pressure sensor 324 with a rigid structure-foam sheet interface between the two. This interface allows the force applied on the PPG sensor 320 (but not elsewhere on the enclosure) to reach the pressure sensor 324 and be uniformly distributed on the pressure sensor 324. The applied pressure by the anatomy of interest is the total force measured by all of the sensing elements divided by the pressing area. The pressure sensor 324 was calibrated as it resides in the sensor by placing high density weights on the PPG sensor 320. The relationship between the measured voltage and known pressure was nearly linear over physiologic pressures.

FIGS. 9A and 9B are graphs depicting the pressure applied 116 to the sensor 108 and the target pressure 118 displayed when pressure is applied to the sensor 108 as well as the blood volume oscillations 120 that are also generated to determine BP. The sensor is connected to the computer via a data acquisition system (NI USB6009, National Instruments, USA). The visual display, which is implemented using the data acquisition system software (LabVIEW, National Instruments), guides the user in performing the actuation against the anatomy of interest, in this example, a finger. The guidance is simply a display of the applied pressure 116 as it evolves in real-time within a plotting window that tells the user to raise the pressure steadily to 150 mmHg over a 30-sec interval but not in any preset way (e.g., a linear rise). The display also illustrates the blood volume oscillations 120 as it is being measured and estimated BP levels. Standard algorithms, which are implemented using computing software (MATLAB, Mathworks, USA), are applied to the measured pressure 116 and the blood volume oscillations 120 to construct an oscillogram 128 and estimate BP at the anatomy of interest. In particular, BP is estimated using the fixed-ratio algorithm with the ratios set to typical values of 0.85 for DP and 0.55 for SP.

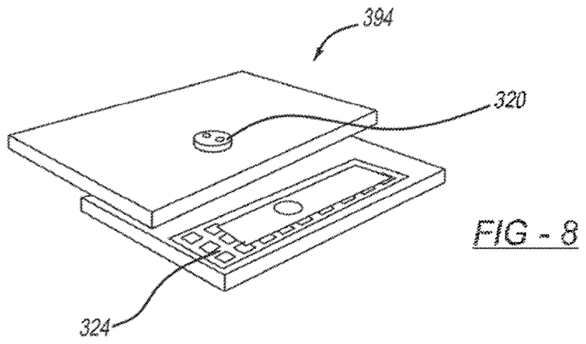

FIG. 10 is a diagram depicting the example of an index finger that exerts the pressure applied 116 to the prototype system 394. The pressing protocol includes pressing the PPG sensor 320 with the center of the index finger, shown in FIG. 10, above the top knuckle 396, which is above the transverse palmer arch artery 400. The protocol further includes instructing the user to apply the force in the normal direction while the finger is at heart level in order to eliminate confounding hydrostatic effects. As previously described, the user may follow the finger pressing protocol through the display 104 of the mobile device 100 using an application to display the necessary graphs and prompts on the display 104. When measuring the BP of the index finger, the traverse palmer arch artery 400 is the target artery, as discussed below.

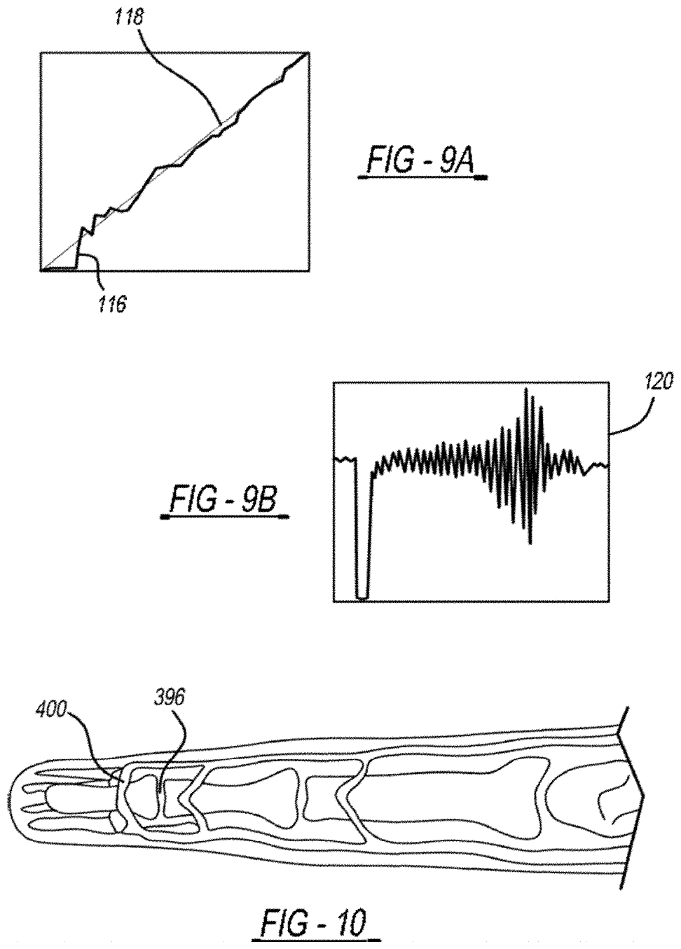

FIGS. 11A-11C are diagrams depicting the results of the prototype system 394. This basic prototype system 394 was studied in human subjects in the seated posture (similar to cuff BP measurements) under IRB approval. Each subject addressed the sensor placed on a table at heart level to eliminate hydrostatic effects. The subject placed the center of her index finger above the top knuckle 396 on the center of the PPG sensor 320. In this way, BP from the transverse palmer arch artery 400 would be targeted for measurement. The subject also rested a portion of her finger below the top knuckle 396 on the sensor enclosure to ensure normal direction force application. The subject then performed the finger actuation under visual guidance. Many people, including those in their 60's, can easily implement the finger actuation on the first try or after one or two practice trials.

The cuff-less BP estimates of the system were compared to BP measurements from an oscillometric arm cuff device (BP760, Omron) in 23 mostly inexperienced students and staff at Michigan State University (MSU). Each subject was allowed to practice the finger actuation a couple of times before recording the BP estimates. FIGS. 11A-11C depict cuff-less BP estimates versus the cuff measurements. FIG. 11A depicts the cuff-less DP versus the cuff DP 404. FIG. 11B depicts the cuff-less MP versus the cuff MP 408. FIG. 11C depicts the cuff-less SP versus the cuff SP 412. The bias and precision errors were about 1-3 and 7-11 mmHg. Since finger SP is larger than Brachial SP, a crude estimate of Brachial SP via a basic formula (SP=2.5*MP−1.5*DP) was instead used here.

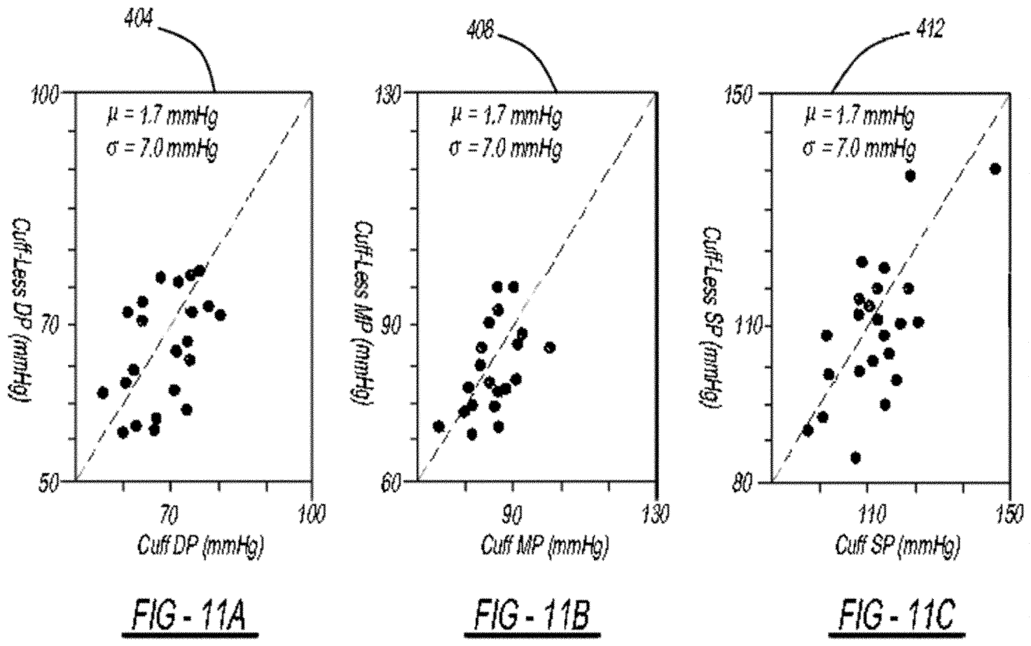

FIG. 12 is a diagram depicting an example embodiment of cuff-less BP measurement devices. FIG. 12 depicts the measurement system included in a computer 500 and a watch 504. The computer 500 and the watch 504 include the sensor 108 and the display 104 as shown in FIG. 1. FIG. 13 is a diagram depicting an example PPG sensor 320 on the sensor 108. To measure the blood volume oscillations, any available sensor known in the art may be employed. As shown in FIG. 13, the PPG sensor 320, which is implemented in pulse oximeters, is used. The reflectance-mode PPG sensor, in particular, may be congruent with most form factors. The green wavelength, which typically yields a high AC signal relative to the DC signal, or a near infrared wavelength, which penetrates beneath the skin, or multiple wavelengths (e.g., red and infrared to also permit measurement of arterial oxygen saturation (SpO2)) may be employed.

A single photodetector 508 and light emitting diode 512 (LED) pair may be used for measurement of blood volume in a target artery 516 such as the transverse palmer arch artery 400 above the top knuckle 396 of an index finger (see FIG. 10). The distance between the LED 512 and photodetector 508 may be a few (e.g., 2) millimeters and positioned in various ways including on a line perpendicular to the flow of arterial blood, as shown in FIG. 13. The arrows of the target artery 516 depict the arterial blood flow.

Alternatively, the PPG sensor 320 may be a transmissive-mode PPG sensor. For example, the PPG sensor 320 can be in a ring or “clothespin” format with the pressure sensor mounted below the photodetector 508. When the user presses their anatomy of interest inside the PPG sensor 320, such as a sensor ring or paddle or other contact onto an external, hard surface, the anatomy of interest deforms. The sensor can be made out of a soft material to enable the proper transmission of the force and keep the LED 512 preloaded onto the top of the finger or other anatomy of interest.

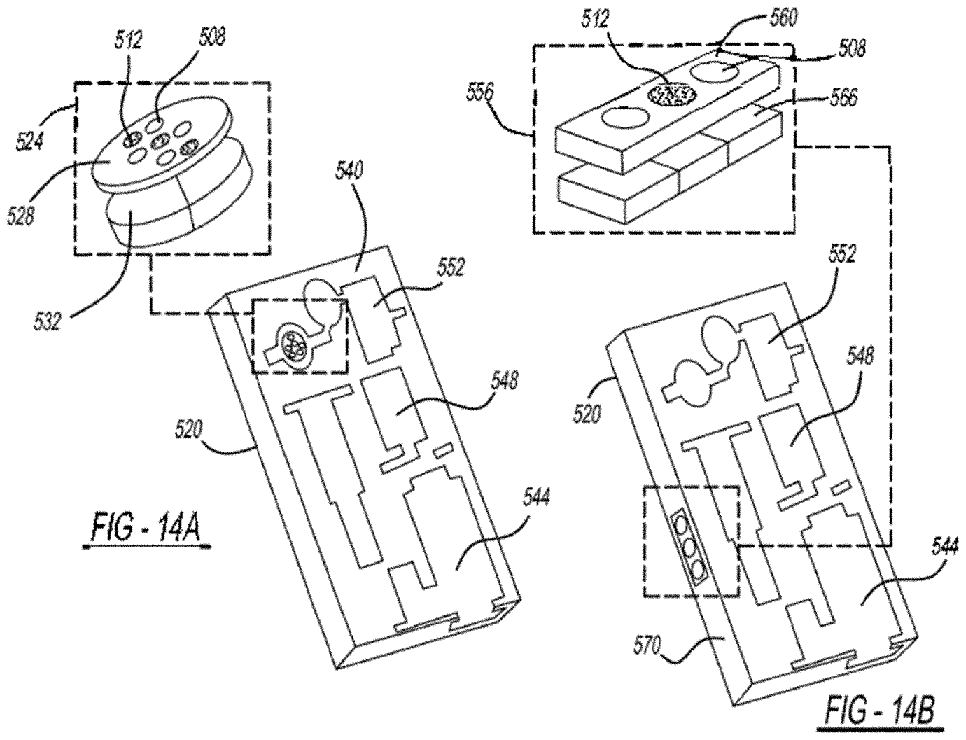

FIGS. 14A and 14B are diagrams depicting example embodiments of sensors integrated into an encasing 520 of mobile devices 100. The encasing 520 of the mobile device 100 is a sleeve designed to enclose the mobile device 100. Cases are commonly used for mobile devices 100 to protect the mobile device 100 from damage such as scratches. In the present disclosure, the encasing 520 is a separate sleeve for the mobile device 100 and includes the necessary components for the BP measurement system. The encasing 520 is designed to interface with the mobile device 100 using a network communication device such as Bluetooth.

In FIG. 14A, a circular sensor 524 is shown. The circular sensor 524 includes a PPG sensor array 528 with multiple light emitting diodes (LEDs) 512 and multiple of photodetectors 508. The PPG sensor array 528 is coupled to a pressure array 532. Alternatively, a single LED 512 may be employed, as exemplified in FIG. 14B. With the PPG sensor array 528, the target artery 516 may be located and blood volume oscillations from therein may be measured via selection of the largest amplitude waveform obtained from the photodetectors 508. The surface of the PPG sensor array 528, which constitutes the pressing area, may be circular with a 10 mm diameter or occupy a similar area with the same or different shape, such as a rectangle 536, as shown in FIG. 14B. The 10 mm area (or other sized or shaped area, as desired), facilitates the actuation by the anatomy of interest in that a high enough pressure can be easily achieved without being overly sensitive to the forces of the anatomy of interest. The PPG sensor array 528 surface may be flat or concave to facilitate positioning of the anatomy of interest on the PPG sensor array 528.

In another embodiment, a red, green, and blue (RGB) camera (e.g. from e-con Systems, USA) can be used as reflectance-mode PPG sensor array. The RGB camera can operate as multiple photodetectors and the camera flash can operate as a light source. Each pixel in a RGB video provides blood volume waveforms at the three wavelengths. That is, the RGB video can construct a “PPG image”. From the PPG image, “hot spots” in the anatomy of interest can be identified to measure the blood volume from the target artery 516. The RBG camera, already built in the mobile device, may be leveraged to measure the blood volume oscillations.

FIG. 14A also depicts the PPG sensor array 528 as coupled to the pressure array 532. Various pressure or force sensors such as resistive, capacitive, or piezoelectric transducers may be included in the pressure array 532 to measure the pressure applied 116 to the sensor 108. The specifications for the pressure sensor should be congruent with the measurement of BP (e.g., a pressure range of O to 300 mmHg and a resolution of about 0.1 mmHg). As discussed above, the pressure sensor 10 may be thin-filmed. In other embodiments, larger sensors such as load cells can be accommodated in certain form factors. The pressure sensor array 532 would typically be the same size as the PPG sensor array 528 surface. A single pressure transducer may be employed or multiple, smaller pressure sensing elements can be used to ensure that the pressure is being uniformly applied via examination of the similarity amongst the forces exerted on each individual sensor. In other words, one of the benefits of using multiple pressure sensors, in any array form, is that the force applied to each individual pressure sensor can be measured to determine whether the force is applied uniformly on the pressure sensors. That is, if the forces applied to each pressure sensor vary amongst one another, the force is not being applied uniformly. The force applied to each pressure sensor can be used to guide a user for successful actuation of the sensor.

As shown in FIG. 14A, the circular sensor 524 is embedded on a back 540 of an encasing 520 for the mobile device 100. The encasing 520 includes a processor 544, a battery 548, and an analog to digital converter 552. The encasing 520 may include a data acquisition system to record measurements of BP in storage already included on the mobile device the encasing 520 holds. The system would include analog signal conditioning (including signal amplification and filtering with, e.g., low and high cutoff frequencies of about 0.5 to at least 10 Hz) followed by analog-to-digital conversion at 552 (with, e.g., a sampling rate of at least 25 Hz and a resolution of 12 bits or more). Alternative to the processor 544 a microcontroller may be included in the encasing 520. The microcontroller may include a Bluetooth transmission module, which may be employed to send the digitized data wirelessly to the mobile device 100 for display and processing. In other embodiments, any network communication device may be used to receive and transmit digitized data between the encasing 520 and the mobile device 100.

The entire “external” system may be battery powered by the battery 548. In form factors built into the mobile device, the digitized data may be sent to the display 104 and stored in an available medium for processing in the mobile device 100. The storage medium, however, may be included in the encasing 520. The necessary components can also be added on to or included in existing mobile devices 100 such as a cell phone, PDAs, laptops, tablets, wearables including smartwatches and wristbands, or any other form of a portable electronic device.

To compute BP or determine that the actuation against the anatomy of interest was unsuccessful, the recorded data, stored in the storage medium, are analyzed by a set of algorithms implemented on the mobile device's processor 544.

The quality of the pressure applied 116 to the sensor 108, e.g., in FIG. 9A, and blood volume waveform, e.g., in FIG. 98, is initially assessed possibly after some filtering (e.g., bandpass filtering with cutoff frequencies of 0.5 to 10 Hz for the blood volume waveform). If the pressure applied 116 to the sensor 108 does not cover a wide enough range (e.g., at least 50 mmHg) over a sufficiently long time interval (e.g., at least 10 seconds) or the shape of the blood volume pulses often do not show physiologic character (i.e., a rapid rise followed by a slower decay), then the actuation by the anatomy of interest may be deemed unsuccessful. In the event that multiple pressure sensors 532 are employed, the actuation by the anatomy of interest can also be deemed unsuccessful, if the pressure experienced by each sensor is significantly different, as previously described. Otherwise, the multiple pressures may be averaged to yield a single pressure applied 116 to the sensor 108.

If the pressure applied 116 to the sensor 108 and blood volume waveform are considered to be of sufficient quality, the oscillogram 128 is constructed, for example by the oscillogram generator 308 of FIG. 3, according to any method known in the art of cuff BP measurement. For example, the pressure applied 116 to the sensor 108 is low-pass filtered or a polynomial is fitted to mitigate spurious fluctuations in the pressure applied 116 to the sensor 108. The maximum and minimum of each beat of the blood volume waveform are detected. These extrema, as a function of the pressure applied 116 to the sensor 108, are median filtered to attenuate respiratory and pulse rate variability as well as artifact. Finally, the extrema are linearly interpolated, and the difference between the two envelopes is taken as the oscillogram 128. If the oscillogram 128 does not exhibit physiologic character (e.g., uni-modal and smooth), the actuation by the anatomy of interest may also be considered unsuccessful. For a physiologic oscillogram, a parametric model (e.g., single or multiple Gaussian functions or a quadratic function) may be fitted to yield a more robust oscillogram.

BP is next estimated from successful oscillograms according to known algorithms in the art of oscillometry. For example, the basic maximum oscillation algorithm, the standard fixed-ratio algorithm seen in FIGS. 6A and 6B, the fixed-slope algorithm, or some combination or variant of the two may be employed. Alternatively, a patient-specific algorithm seen in FIGS. 7A and 7B, which may be more accurate than conventional population-based algorithms, may be applied. A third option is a combination of simple and more sophisticated algorithms. For example, a combined algorithm can output a MP estimate via the pressure applied 116 at which the oscillogram 128 is maximal when relatively low quality oscillograms are obtained and all three BP estimates via the patient-specific algorithm when higher quality oscillograms are measured. The algorithm can also output a confidence level on the accuracy of its BP estimates based on the measurement quality.

Standard Brachial (arm) BP, which is the proven cardiovascular risk factor, may also be derived. For example, while finger and Brachial MP and DP are similar, finger SP is higher than Brachial SP due to arterial wave reflection. Brachial SP may be estimated by simple transformations of finger BP. For example, since the ratio of finger SP to Brachial SP may decrease with age, an age-dependent scaling of finger SP can be applied to estimate Brachial SP. Alternatively, a transfer function may be applied to more accurately estimate Brachial BP from finger BP. The transfer function would require input of the finger BP waveform, which can be obtained with the patient-specific algorithm. Another possibility is to estimate Brachial SP from finger DP and MP using empirical formulas designed for Brachial BP (e.g., MP=(⅓)*SP+(⅔)*DP). As will be appreciated, the Brachial SP may be estimated by analogous transformations of the BP measured at other arterial locations, such as the superficial and deeper arteries of any part of the body including the digital and other arteries of the hand, thumb, nose (inside or outside), forehead, scalp, ear, toe, wrist (radial artery), arm (brachial, axillary, subclavian, or ulnar) arteries, lower extremities (femoral, popliteal, tibial, or dorsalis pedis) arteries, or the trunk (aorta or ileac) arteries.

Other physiologic parameters of interest such as pulse rate and pulse rate variability may also be computed from the blood volume waveform using any method known in the art. The pulse rate variability can be assessed to determine the presence of an arrhythmia such as atrial fibrillation using any method known in the art. If red and infrared PPG measurements are available, SpO2 may additionally be computed using an existing method.

Finally, an algorithm can also be employed for early termination of the actuation against the anatomy of interest. For example, the oscillogram 128 can be constructed in real-time as the pressure is being applied against the anatomy of interest. If the portion of the oscillogram that has been currently constructed is similar to the same portion of a previously constructed, complete oscillogram, then the previous BP levels can reasonably be assumed and immediately outputted. In this way, some BP measurements may only take a few seconds to make.

FIG. 14B is a diagram depicting a rectangular sensor 556. The rectangular sensor 556 includes a rectangular PPG sensor array 560 with multiple photodetectors 508 and one LED 512 and a rectangular pressure sensor array 566. The rectangular sensor 556 is placed on a side 570 of the encasing 520, which may be better suited to the finger pressing task, or for pressing against other anatomical structures as taught herein.

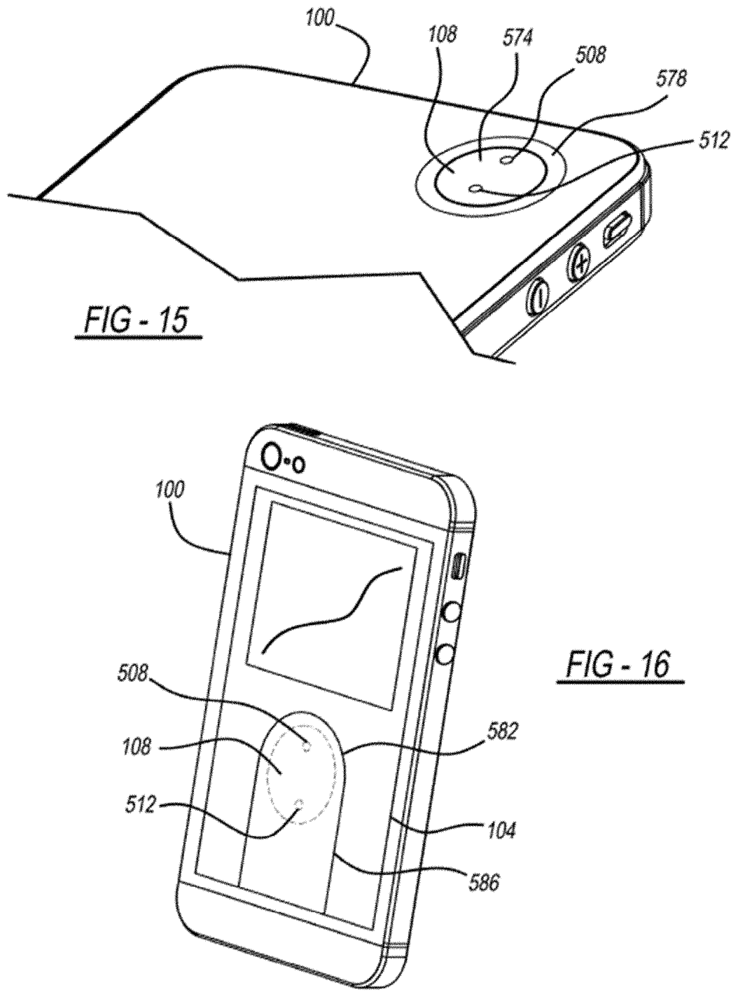

FIG. 15 is a diagram depicting another embodiment of the cuff-less BP measurement system on the mobile device 100. For example, the mobile device 100 depicted in FIG. 15 is the same as the mobile device 100 of FIG. 1. However, instead of using the encasing 520 to couple the sensor 108 to the mobile device 100, the existing PPG sensor or the existing RGB camera 574 on the mobile device 100 is used. To complete the sensor, a donut shaped pressure sensor 578 is placed on top of and around the PPG sensor 574 to allow the passage of light.

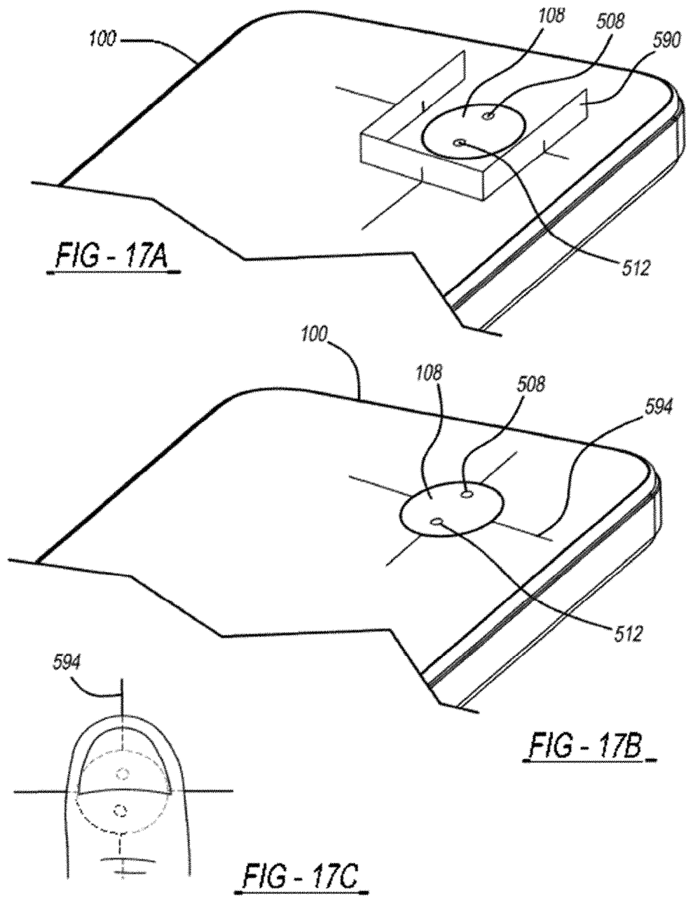

FIG. 16 is a diagram depicting another embodiment of the cuff-less BP measurement system. FIG. 16 depicts the cuff-less BP measurement system with an infrared PPG sensor 582 located below the display 104 of the mobile device 100. The sensor 108, employing the infrared PPG sensor 582, can be placed under the display 104 while leveraging the pressure sensing capabilities of the display 104. In this case, a picture of the user's finger 586 can be displayed indicating exactly where the user should position their finger for subsequent pressing. However, it will be appreciated that this process can be performed for measuring local BP at other anatomies of interest.

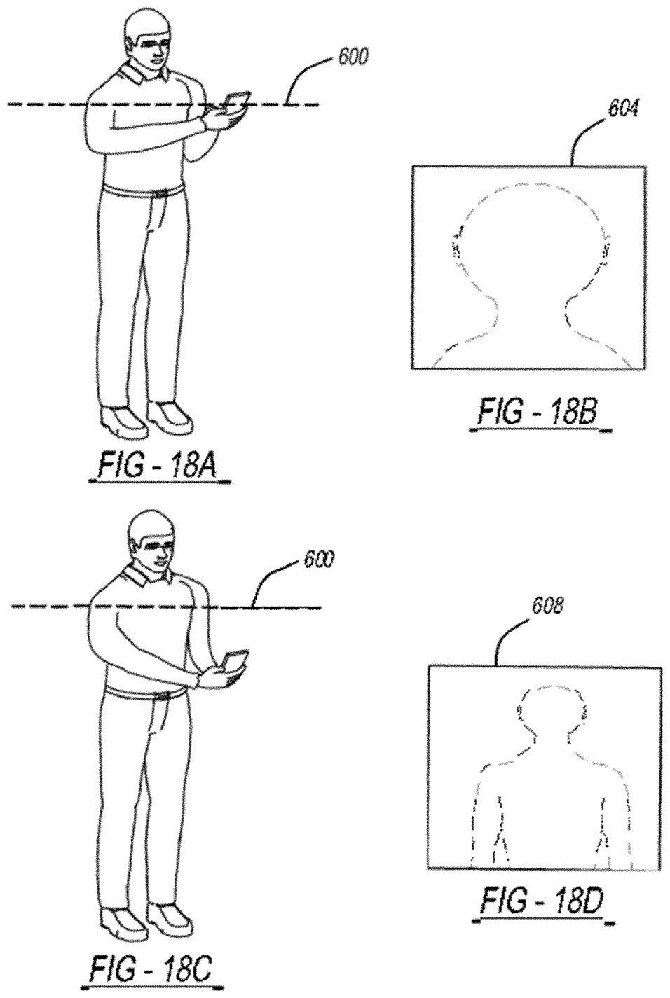

FIGS. 17A-17C are diagrams depicting an embodiment of the cuff-less BP measurement system with an illustrative example of finger placement indicators. In FIG. 17A, the finger placement indicator 590 facilitates the finger positioning on the sensor 108 coupled to the mobile device 100. The finger placement indicator 590 is a physical barrier placed around the sensor 108 to guide repeatable finger placement. The finger placement indicator 590 may be included on the encasing 520 or as a separate item attached to the mobile device 100 when using the RGB camera or PPG sensor of the mobile device 100. The finger placement indicator 590 can be adjustable to accommodate different finger sizes or multiple finger placement indicators 590 can be offered for different finger sizes (i.e., small, medium, and large). The anatomical placement indicator can also be a more subtle physical barrier than that indicated 590. Likewise, it will be appreciated that this process can be modified for measuring local BP at other anatomies of interest.

In FIG. 17B, a visual guide placement indicator 594 (or for placement other anatomy of interest) is placed on the mobile device 100. Alternatively, the visual guide placement indicator 594 may be on the encasing 520 of the mobile device 100. For example, lines can be drawn over the sensor 108 to guide the user in placing the base of the finger nail between the LED 512 and photodetector 508 and the center of the finger on the line passing through the LED 512 and photodetector 508, as also shown in FIG. 17C at 594. In addition, the sensor 108 may be positioned so that a portion of the finger below the top knuckle can also rest on the device to ensure normal direction force application.

The cuff-less BP measurement system, in any of the embodiments, may also be accompanied by additional means to guide proper actuation by the anatomy of interest. In particular, proper placement of the anatomy of interest for a specific user may be determined via an initialization protocol. This protocol involves measuring the oscillogram 128 at different positions on the sensor 108 and choosing the anatomical position based on the oscillogram amplitude and morphology (e.g., maximal oscillogram) or based on an initial cuff BP reading.

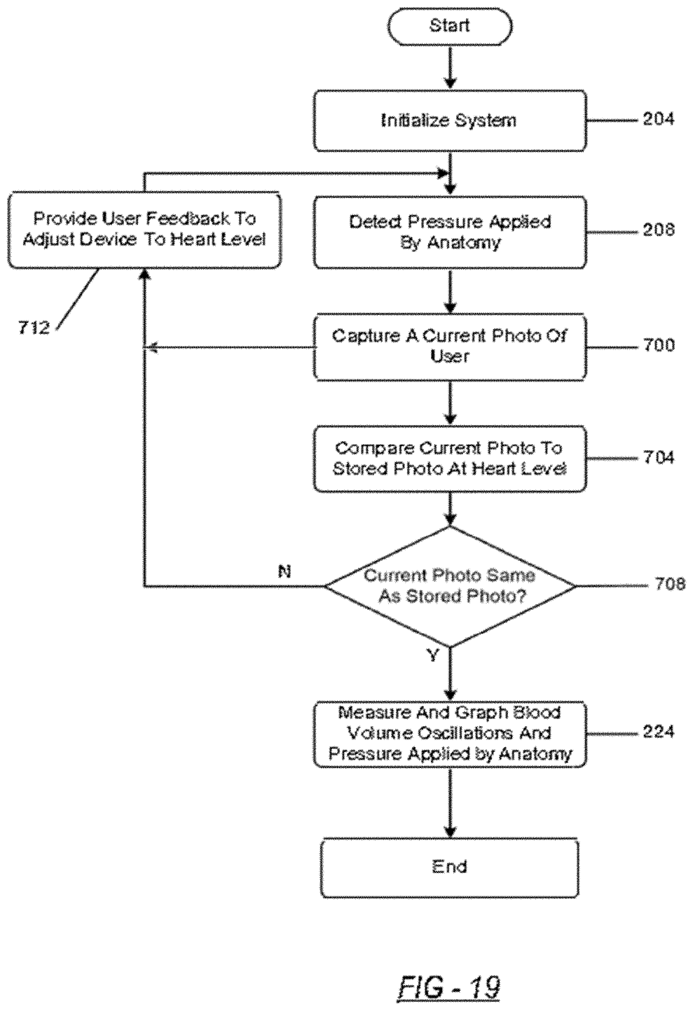

FIGS. 18A-18D are diagrams depicting an example position detection system included in the cuff-less BP measurement system. The camera that is already built in the mobile device 100 may be leveraged to ensure that the mobile device 100 is being held at heart level 600. The camera of the mobile device 100, as discussed above with respect to the RGB camera, may be used as a PPG sensor as well. For position detection purposes, the camera records an image of the face 604 while the mobile device 100 is known to be at heart level 600. For subsequent BP measurements, the camera records another image and compares the current image to the previously recorded image 604 known to be at heart level 600. The silhouette from the current image should be close enough to that of the previously recorded image. Otherwise, the application asks the user to put the mobile device at heart level. An example image when the mobile device 100 is below heart level 600 is shown at 608. This approach would only be effective when the user is in the upright posture. The accelerometer that is already in the mobile device 100 can also possibly be leveraged for confirming heart level 600.

In another embodiment, the cuff-less BP system may compensate for BP calculations when it has been detected that the user had their BP measured without holding the mobile device 100 at heart level. For example, after instructing the user to hold the mobile device 100 at heart level and then proceed to steadily increase the applied anatomical pressure, the system measures and records BP measurements. If the system detects that the mobile device 100 is not being held at heart level, then the recorded BP measurements can be adjusted accordingly for the height at which the measurements were being taken using a rho-g-h correction, where rho is the known blood density, g is gravity, and h is the vertical distance between the anatomy of interest and heart estimated from the images.

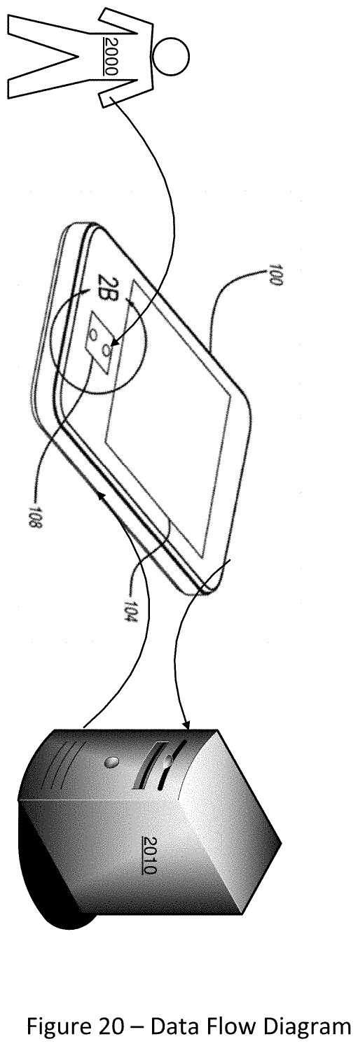

FIG. 19 is a flowchart depicting the example position detection system included in the cuff-less BP measurement system. The system is initialized at 204 and the pressure applied 116 by the anatomy of interest is detected at 208. To ensure that the user is holding the mobile device 100 at heart level, the system captures a current photo 608 of the user at 700. The system then compares the current photo 608 to a previously recorded or stored photo 604 of the user when the mobile device 100 is known to be held at heart level at 704. If the current photo 608 is the same as or similar enough to the previously recorded photo 604, then the system continues to measure and graph blood volume oscillations and pressure applied 116 to the sensor 108 with the anatomy of interest at 224. If, however, the current photo 608 differs from the previously recorded photo 604 by a predetermined amount, then the system provides feedback to the user instructing to user to adjust the mobile device 100 to heart level at 712. In an embodiment, the photos that are captured may be silhouettes or outlines to be able to compare a size of the user to determine if the mobile device 100 is at heart level.

As another example to guide proper anatomical actuation, a skin print, image, or other surface impression, can be obtained and used to confirm and/or guide proper anatomical positioning on the sensor 108 as well as to identify the user for a multi-user device. Maintaining an identity of users ensures measurements are transmitted to the appropriate place. In addition, the application can include an instructional video to explain how to use the device correctly. Alternatively, the user can test for the best anatomical positioning by measuring the user's BP multiple times and recording the anatomical position each time. After at least two attempts to measure the user's BP, the application can determine which BP measurement results in the largest oscillogram and indicate to the user that the recorded anatomical position for the largest oscillogram is the preferred anatomical position for that user.

In other embodiments, the mobile device 100 can act as the actuator instead of the user. For example, the mobile device 100 can include a motor driven system or a mechanical spring that would automatically apply the pressure to the anatomy of interest placed on the sensor 108. Additionally, the method can also be integrated within non-mobile device form factors including elevator control panels, video game controllers, doorbells, keychains, steering wheels, bathroom mirrors, pill bottle caps, etc.

### Example—DIGI™ Data Information System and Controller

FIG. 21 illustrates inventive aspects of a DIGI™ controller 701 for controlling a system such as that illustrated in FIG. 20 implementing some of the embodiments disclosed herein. In this embodiment, the DIGI™ controller 701 may serve to aggregate, process, store, search, serve, identify, instruct, generate, match, and/or facilitate interactions with a computer through various technologies, and/or other related data. In particular, it is contemplated that users of blood pressure monitoring devices as described herein can upload and back up their blood pressure data to a computer database system where the data can be stored retrieved and analyzed for various purposes, such as central monitoring, and conducting studies, provided that appropriate legal privacy requirements are met concerning medical data.

Typically, a user or users, e.g., 733a, which may be people or groups of users and/or other systems, may engage information technology systems (e.g., computers) to facilitate operation of the system and information processing. In turn, computers employ processors to process information; such processors 703 may be referred to as central processing units (CPU). One form of processor is referred to as a microprocessor. CPUs use communicative circuits to pass binary encoded signals acting as instructions to enable various operations. These instructions may be operational and/or data instructions containing and/or referencing other instructions and data in various processor accessible and operable areas of memory 729 (e.g., registers, cache memory, random access memory, etc.). Such communicative instructions may be stored and/or transmitted in batches (e.g., batches of instructions) as programs and/or data components to facilitate desired operations. These stored instruction codes, e.g., programs, may engage the CPU circuit components and other motherboard and/or system components to perform desired operations. One type of program is a computer operating system, which, may be executed by CPU on a computer; the operating system enables and facilitates users to access and operate computer information technology and resources. Some resources that may be employed in information technology systems include: input and output mechanisms through which data may pass into and out of a computer; memory storage into which data may be saved; and processors by which information may be processed. These information technology systems may be used to collect data for later retrieval, analysis, and manipulation, which may be facilitated through a database program. These information technology systems provide interfaces that allow users to access and operate various system components.

In one embodiment, the DIGI™ controller 701 may be connected to and/or communicate with entities such as, but not limited to: one or more users from user input devices 711; peripheral devices 712, user devices, servers; an optional cryptographic processor device 728; and/or a communications network 713. For example, the DIGI™ controller 701 may be connected to and/or communicate with users, e.g., 733a, operating client device(s), e.g., 733b, including, but not limited to, personal computer(s), server(s) and/or various mobile device(s) including, but not limited to, cellular telephone(s), smartphone(s) (e.g., iPhone®, Blackberry®, Android OS-based phones etc.), tablet computer(s) (e.g., Apple iPad™, HP Slate™, Motorola Xoom™, etc.), eBook reader(s) (e.g., Amazon Kindle™, Barnes and Noble's Nook™ eReader, etc.), laptop computer(s), notebook(s), netbook(s), gaming console(s) (e.g., XBOX Live™, Nintendo® DS, Sony PlayStation® Portable, etc.), portable scanner(s) and/or the like.

Networks are commonly thought to comprise the interconnection and interoperation of clients, servers, and intermediary nodes in a graph topology. It should be noted that the term “server” as used throughout this application refers generally to a computer, other device, program, or combination thereof that processes and responds to the requests of remote users across a communications network. Servers serve their information to requesting “clients.” The term “client” as used herein refers generally to a computer, program, other device, user and/or combination thereof that is capable of processing and making requests and obtaining and processing any responses from servers across a communications network. A computer, other device, program, or combination thereof that facilitates, processes information and requests, and/or furthers the passage of information from a source user to a destination user is commonly referred to as a “node.” Networks are generally thought to facilitate the transfer of information from source points to destinations. A node specifically tasked with furthering the passage of information from a source to a destination is commonly called a “router.” There are many forms of networks such as Local Area Networks (LANs), Pico networks, Wide Area Networks (WANs), Wireless Networks (WLANs), etc. For example, the Internet is generally accepted as being an interconnection of a multitude of networks whereby remote clients and servers may access and interoperate with one another.

The DIGI™ controller 701 may be based on computer systems that may comprise, but are not limited to, components such as: a computer systemization 702 connected to memory 729.

### Computer Systemization

A computer systemization 702 may comprise a clock 730, central processing unit (“CPU(s)” and/or “processor(s)” (these terms are used interchangeable throughout the disclosure unless noted to the contrary)) 703, a memory 729 (e.g., a read only memory (ROM) 706, a random access memory (RAM) 705, etc.), and/or an interface bus 707, and most frequently, although not necessarily, are all interconnected and/or communicating through a system bus 704 on one or more (mother)board(s) 702 having conductive and/or otherwise transportive circuit pathways through which instructions (e.g., binary encoded signals) may travel to effect communications, operations, storage, etc. Optionally, the computer systemization may be connected to an internal power source 786; e.g., optionally the power source may be internal. Optionally, a cryptographic processor 726 and/or transceivers (e.g., ICs) 774 may be connected to the system bus. In another embodiment, the cryptographic processor and/or transceivers may be connected as either internal and/or external peripheral devices 712 via the interface bus I/O. In turn, the transceivers may be connected to antenna(s) 775, thereby effectuating wireless transmission and reception of various communication and/or sensor protocols; for example the antenna(s) may connect to: a Texas Instruments WiLink WL1283 transceiver chip (e.g., providing 802.11n, Bluetooth 3.0, FM, global positioning system (GPS) (thereby allowing DIGI™ controller to determine its location)); Broadcom BCM4329FKUBG transceiver chip (e.g., providing 802.11n, Bluetooth 2.1+EDR, FM, etc.); a Broadcom BCM4750IUB8 receiver chip (e.g., GPS); an Infineon Technologies X-Gold 618-PMB9800 (e.g., providing 2G/3G HSDPA/HSUPA communications); and/or the like. The system clock typically has a crystal oscillator and generates a base signal through the computer systemization's circuit pathways. The clock is typically coupled to the system bus and various clock multipliers that will increase or decrease the base operating frequency for other components interconnected in the computer systemization. The clock and various components in a computer systemization drive signals embodying information throughout the system. Such transmission and reception of instructions embodying information throughout a computer systemization may be commonly referred to as communications. These communicative instructions may further be transmitted, received, and the cause of return and/or reply communications beyond the instant computer systemization to: communications networks, input devices, other computer systemizations, peripheral devices, and/or the like. Of course, any of the above components may be connected directly to one another, connected to the CPU, and/or organized in numerous variations employed as exemplified by various computer systems.

The CPU comprises at least one high-speed data processor adequate to execute program components for executing user and/or system-generated requests. Often, the processors themselves will incorporate various specialized processing units, such as, but not limited to: integrated system (bus) controllers, memory management control units, floating point units, and even specialized processing sub-units like graphics processing units, digital signal processing units, and/or the like. Additionally, processors may include internal fast access addressable memory, and be capable of mapping and addressing memory 729 beyond the processor itself; internal memory may include, but is not limited to: fast registers, various levels of cache memory (e.g., level 1, 2, 3, etc.), RAM, etc. The processor may access this memory through the use of a memory address space that is accessible via instruction address, which the processor can construct and decode allowing it to access a circuit path to a specific memory address space having a memory state. The CPU may be a microprocessor such as: AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s). The CPU interacts with memory through instruction passing through conductive and/or transportive conduits (e.g., (printed) electronic and/or optic circuits) to execute stored instructions (i.e., program code) according to conventional data processing techniques. Such instruction passing facilitates communication within the DIGI™ controller and beyond through various interfaces. Should processing requirements dictate a greater amount speed and/or capacity, distributed processors (e.g., Distributed DIGI™ embodiments), mainframe, multi-core, parallel, and/or super-computer architectures may similarly be employed. Alternatively, should deployment requirements dictate greater portability, smaller Personal Digital Assistants (PDAs) may be employed.

Depending on the particular implementation, features of the DIGI™ implementations may be achieved by implementing a microcontroller such as CAST's R8051XC2 microcontroller; Intel's MCS 51 (i.e., 8051 microcontroller); and/or the like. Also, to implement certain features of the DIGI™ embodiments, some feature implementations may rely on embedded components, such as: Application-Specific Integrated Circuit (“ASIC”), Digital Signal Processing (“DSP”), Field Programmable Gate Array (“FPGA”), and/or the like embedded technology. For example, any of the DIGI™ component collection (distributed or otherwise) and/or features may be implemented via the microprocessor and/or via embedded components; e.g., via ASIC, coprocessor, DSP, FPGA, and/or the like. Alternately, some implementations of the DIGI™ may be implemented with embedded components that are configured and used to achieve a variety of features or signal processing.

Depending on the particular implementation, the embedded components may include software solutions, hardware solutions, and/or some combination of both hardware/software solutions. For example, DIGI™ features discussed herein may be achieved through implementing FPGAs, which are a semiconductor devices containing programmable logic components called “logic blocks”, and programmable interconnects, such as the high performance FPGA Virtex series and/or the low cost Spartan series manufactured by Xilinx. Logic blocks and interconnects can be programmed by the customer or designer, after the FPGA is manufactured, to implement any of the DIGI™ features. A hierarchy of programmable interconnects allow logic blocks to be interconnected as needed by the DIGI™ system designer/administrator, somewhat like a one-chip programmable breadboard. An FPGA's logic blocks can be programmed to perform the function of basic logic gates such as AND, and XOR, or more complex combinational functions such as decoders or simple mathematical functions. In most FPGAs, the logic blocks also include memory elements, which may be simple flip-flops or more complete blocks of memory. In some circumstances, the DIGI™ may be developed on regular FPGAs and then migrated into a fixed version that more resembles ASIC implementations. Alternate or coordinating implementations may migrate DIGI™ controller features to a final ASIC instead of or in addition to FPGAs. Depending on the implementation all of the aforementioned embedded components and microprocessors may be considered the “CPU” and/or “processor” for the DIGI™.

### Power Source

The power source 786 may be of any standard form for powering small electronic circuit board devices such as the following power cells: alkaline, lithium hydride, lithium ion, lithium polymer, nickel cadmium, solar cells, and/or the like. Other types of AC or DC power sources may be used as well. In the case of solar cells, in one embodiment, the case provides an aperture through which the solar cell may capture photonic energy. The power cell 786 is connected to at least one of the interconnected subsequent components of the DIGI™ thereby providing an electric current to all subsequent components. In one example, the power source 786 is connected to the system bus component 704. In an alternative embodiment, an outside power source 786 is provided through a connection across the I/O 708 interface. For example, a USB and/or IEEE 1394 connection carries both data and power across the connection and is therefore a suitable source of power.

### Interface Adapters

Interface bus(ses) 707 may accept, connect, and/or communicate to a number of interface adapters, conventionally although not necessarily in the form of adapter cards, such as but not limited to: input output interfaces (I/O) 708, storage interfaces 709, network interfaces 710, and/or the like. Optionally, cryptographic processor interfaces 727 similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and/or the like.

Storage interfaces 709 may accept, communicate, and/or connect to a number of storage devices such as, but not limited to: storage devices 714, removable disc devices, and/or the like. Storage interfaces may employ connection protocols such as, but not limited to: (Ultra) (Serial) Advanced Technology Attachment (Packet Interface) ((Ultra) (Serial) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces 710 may accept, communicate, and/or connect to a communications network 713. Through a communications network 713, the DIGI™ controller is accessible through remote clients 733b (e.g., computers with web browsers) by users 733a. Network interfaces may employ connection protocols such as, but not limited to: direct connect, Ethernet (thick, thin, twisted pair 10/100/1000 Base T, and/or the like), Token Ring, wireless connection such as IEEE 802.11a-x, and/or the like. Should processing requirements dictate a greater amount speed and/or capacity, distributed network controllers (e.g., Distributed DIGI™), architectures may similarly be employed to pool, load balance, and/or otherwise increase the communicative bandwidth required by the DIGI™ controller. A communications network may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface. Further, multiple network interfaces 710 may be used to engage with various communications network types 713. For example, multiple network interfaces may be employed to allow for the communication over broadcast, multicast, and/or unicast networks.

Input Output interfaces (I/O) 708 may accept, communicate, and/or connect to user input devices 711, peripheral devices 712, cryptographic processor devices 728, and/or the like. I/O may employ connection protocols such as, but not limited to: audio: analog, digital, monaural, RCA, stereo, and/or the like; data: Apple Desktop Bus (ADB), IEEE 1394a-b, serial, universal serial bus (USB); infrared; joystick; keyboard; midi; optical; PC AT; PS/2; parallel; radio; video interface: Apple Desktop Connector (ADC), BNC, coaxial, component, composite, digital, Digital Visual Interface (DVI), high-definition multimedia interface (HDMI), RCA, RF antennae, S-Video, VGA, and/or the like; wireless transceivers: 802.11a/b/g/n/x; Bluetooth; cellular (e.g., code division multiple access (CDMA), high speed packet access (HSPA(+)), high-speed downlink packet access (HSDPA), global system for mobile communications (GSM), long term evolution (LTE), WiMax, etc.); and/or the like. One typical output device may include a video display, which typically comprises a Cathode Ray Tube (CRT) or Liquid Crystal Display (LCD) based monitor with an interface (e.g., DVI circuitry and cable) that accepts signals from a video interface, may be used. The video interface composites information generated by a computer systemization and generates video signals based on the composited information in a video memory frame. Another output device is a television set, which accepts signals from a video interface. Typically, the video interface provides the composited video information through a video connection interface that accepts a video display interface (e.g., an RCA composite video connector accepting an RCA composite video cable; a DVI connector accepting a DVI display cable, etc.).

User input devices 711 often are a type of peripheral device 712 (see below) and may include: card readers, dongles, finger print readers, gloves, graphics tablets, joysticks, keyboards, microphones, mouse (mice), remote controls, retina readers, touch screens (e.g., capacitive, resistive, etc.), trackballs, trackpads, sensors (e.g., accelerometers, ambient light, GPS, gyroscopes, proximity, etc.), styluses, and/or the like.

Peripheral devices 712, such as user devices 100 or other components of the system, such as peripheral sensors and the like, may be connected and/or communicate to I/O and/or other facilities of the like such as network interfaces, storage interfaces, directly to the interface bus, system bus, the CPU, and/or the like. Peripheral devices may be external, internal and/or part of the DIGI™ controller.

Cryptographic units such as, but not limited to, microcontrollers, processors 726, interfaces 727, and/or devices 728 may be attached, and/or communicate with the DIGI™ controller. A MC68HC16 microcontroller, manufactured by Motorola Inc., may be used for and/or within cryptographic units. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Equivalent microcontrollers and/or processors may also be used. Other commercially available specialized cryptographic processors include: the Broadcom's CryptoNetX and other Security Processors; nCipher's nShield, SafeNet's Luna PCI (e.g., 7100) series; Semaphore Communications' 40 MHz Roadrunner 184; Sun's Cryptographic Accelerators (e.g., Accelerator 6000 PCIe Board, Accelerator 500 Daughtercard); Via Nano Processor (e.g., L2100, L2200, U2400) line, which is capable of performing 500+ MB/s of cryptographic instructions; VLSI Technology's 33 MHz 6868; and/or the like.

### Memory

Generally, any mechanization and/or embodiment allowing a processor to affect the storage and/or retrieval of information is regarded as memory 729. However, memory is a fungible technology and resource, thus, any number of memory embodiments may be employed in lieu of or in concert with one another. It is to be understood that the DIGI™ controller and/or a computer systemization may employ various forms of memory 729. For example, a computer systemization may be configured wherein the functionality of on-chip CPU memory (e.g., registers), RAM, ROM, and any other storage devices are provided by a paper punch tape or paper punch card mechanism; of course such an embodiment would result in an extremely slow rate of operation. In a typical configuration, memory 729 will include ROM 706, RAM 705, and a storage device 714. A storage device 714 may be any conventional computer system storage. Storage devices may include a drum; a (fixed and/or removable) magnetic disk drive; a magneto-optical drive; an optical drive (i.e., Blueray, CD ROM/RAM/Recordable (R)/ReWritable (RW), DVD R/RW, HD DVD R/RW etc.); an array of devices (e.g., Redundant Array of Independent Disks (RAID)); solid state memory devices (USB memory, solid state drives (SSD), etc.); other processor-readable storage mediums; and/or other devices of the like. Thus, a computer systemization generally requires and makes use of memory.

### Component Collection

The memory 729 may contain a collection of program and/or database components and/or data such as, but not limited to: operating system component(s) 715 (operating system); information server component(s) 716 (information server); user interface component(s) 717 (user interface); Web browser component(s) 718 (Web browser); database(s) 719; mail server component(s) 721; mail client component(s) 722; cryptographic server component(s) 720 (cryptographic server) and/or the like (i.e., collectively a component collection). These components may be stored and accessed from the storage devices and/or from storage devices accessible through an interface bus. Although non-conventional program components such as those in the component collection, typically, are stored in a local storage device 714, they may also be loaded and/or stored in memory such as: peripheral devices, RAM, remote storage facilities through a communications network, ROM, various forms of memory, and/or the like.

### Operating System

The operating system component 715 is an executable program component facilitating the operation of the DIGI™ controller. Typically, the operating system facilitates access of I/O, network interfaces, peripheral devices, storage devices, and/or the like. The operating system may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/MilleniumNT/Vista/XP (Server), Palm OS, and/or the like. An operating system may communicate to and/or with other components in a component collection, including itself, and/or the like. Most frequently, the operating system communicates with other program components, user interfaces, and/or the like. For example, the operating system may contain, communicate, generate, obtain, and/or provide program component, system, user, and/or data communications, requests, and/or responses. The operating system, once executed by the CPU, may enable the interaction with communications networks, data, I/O, peripheral devices, program components, memory, user input devices, and/or the like. The operating system may provide communications protocols that allow the DIGI™ controller to communicate with other entities through a communications network 713. Various communication protocols may be used by the DIGI™ controller as a subcarrier transport mechanism for interaction, such as, but not limited to: multicast, TCP/IP, UDP, unicast, and/or the like.

### Information Server

An information server component 716 is a stored program component that is executed by a CPU. The information server may be a conventional Internet information server such as, but not limited to Apache Software Foundation's Apache, Microsoft's Internet Information Server, and/or the like. The information server may allow for the execution of program components through facilities such as Active Server Page (ASP), ActiveX, (ANSI) (Objective-) C (++), C# and/or .NET, Common Gateway Interface (CGI) scripts, dynamic (D) hypertext markup language (HTML), FLASH, Java, JavaScript, Practical Extraction Report Language (PERL), Hypertext Pre-Processor (PHP), pipes, Python, wireless application protocol (WAP), WebObjects, and/or the like. The information server may support secure communications protocols such as, but not limited to, File Transfer Protocol (FTP); HyperText Transfer Protocol (HTTP); Secure Hypertext Transfer Protocol (HTTPS), Secure Socket Layer (SSL), messaging protocols (e.g., America Online (AOL) Instant Messenger (AIM), Application Exchange (APEX), ICQ, Internet Relay Chat (IRC), Microsoft Network (MSN) Messenger Service, Presence and Instant Messaging Protocol (PRIM), Internet Engineering Task Force's (IETF's) Session Initiation Protocol (SIP), SIP for Instant Messaging and Presence Leveraging Extensions (SIMPLE), open XML-based Extensible Messaging and Presence Protocol (XMPP) (i.e., Jabber or Open Mobile Alliance's (OMA's) Instant Messaging and Presence Service (IMPS)), Yahoo! Instant Messenger Service, and/or the like. The information server provides results in the form of Web pages to Web browsers, and allows for the manipulated generation of the Web pages through interaction with other program components. After a Domain Name System (DNS) resolution portion of an HTTP request is resolved to a particular information server, the information server resolves requests for information at specified locations on the DIGI™ controller based on the remainder of the HTTP request. For example, a request such as http://123.124.125.126/myInformation.html might have the IP portion of the request “123.124.125.126” resolved by a DNS server to an information server at that IP address; that information server might in turn further parse the http request for the “/myInformation.html” portion of the request and resolve it to a location in memory containing the information “myInformation.html.” Additionally, other information serving protocols may be employed across various ports, e.g., FTP communications across port 21, and/or the like. An information server may communicate to and/or with other components in a component collection, including itself, and/or facilities of the like. Most frequently, the information server communicates with the DIGI™ database 719, operating systems, other program components, user interfaces, Web browsers, and/or the like.

Access to the DIGI™ database may be achieved through a number of database bridge mechanisms such as through scripting languages as enumerated below (e.g., CGI) and through inter-application communication channels as enumerated below (e.g., CORBA, WebObjects, etc.). Any data requests through a Web browser are parsed through the bridge mechanism into appropriate grammars as required by the DIGI™. In one embodiment, the information server would provide a Web form accessible by a Web browser. Entries made into supplied fields in the Web form are tagged as having been entered into the particular fields, and parsed as such. The entered terms are then passed along with the field tags, which act to instruct the parser to generate queries directed to appropriate tables and/or fields. In one embodiment, the parser may generate queries in standard SQL by instantiating a search string with the proper join/select commands based on the tagged text entries, wherein the resulting command is provided over the bridge mechanism to the DIGI™ as a query. Upon generating query results from the query, the results are passed over the bridge mechanism, and may be parsed for formatting and generation of a new results Web page by the bridge mechanism. Such a new results Web page is then provided to the information server, which may supply it to the requesting Web browser.

Also, an information server may contain, communicate, generate, obtain, and/or provide program component, system, user, and/or data communications, requests, and/or responses.

### User Interface

Computer interfaces in some respects are similar to automobile operation interfaces. Automobile operation interface elements such as steering wheels, gearshifts, and speedometers facilitate the access, operation, and display of automobile resources, and status. Computer interaction interface elements such as check boxes, cursors, menus, scrollers, and windows (collectively and commonly referred to as widgets) similarly facilitate the access, capabilities, operation, and display of data and computer hardware and operating system resources, and status. Operation interfaces are commonly called user interfaces. Graphical user interfaces (GUIs) such as the Apple Macintosh Operating System's Aqua, IBM's OS/2, Microsoft's Windows 2000/2003/3.1/95/98/CE/Millenium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, Yahoo! User Interface, any of which may be used and) provide a baseline and means of accessing and displaying information graphically to users.

A user interface component 717 is a stored program component that is executed by a CPU. The user interface may be a conventional graphic user interface as provided by, with, and/or atop operating systems and/or operating environments such as already discussed. The user interface may allow for the display, execution, interaction, manipulation, and/or operation of program components and/or system facilities through textual and/or graphical facilities. The user interface provides a facility through which users may affect, interact, and/or operate a computer system. A user interface may communicate to and/or with other components in a component collection, including itself, and/or facilities of the like. Most frequently, the user interface communicates with operating systems, other program components, and/or the like. The user interface may contain, communicate, generate, obtain, and/or provide program component, system, user, and/or data communications, requests, and/or responses.

### Web Browser

A Web browser component 718 is a stored program component that is executed by a CPU. The Web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices. A Web browser may communicate to and/or with other components in a component collection, including itself, and/or facilities of the like. Most frequently, the Web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a Web browser and information server, a combined application may be developed to perform similar functions of both. The combined application would similarly affect the obtaining and the provision of information to users, user agents, and/or the like from the DIGI™ enabled nodes. The combined application may be nugatory on systems employing standard Web browsers.

### Mail Server

A mail server component 721 is a stored program component that is executed by a CPU 703. The mail server may be a conventional Internet mail server such as, but not limited to sendmail, Microsoft Exchange, and/or the like. The mail server may allow for the execution of program components through facilities such as ASP, ActiveX, (ANSI) (Objective-) C (++), C# and/or .NET, CGI scripts, Java, JavaScript, PERL, PHP, pipes, Python, WebObjects, and/or the like. The mail server may support communications protocols such as, but not limited to: Internet message access protocol (IMAP), Messaging Application Programming Interface (MAPI)/Microsoft Exchange, post office protocol (POP3), simple mail transfer protocol (SMTP), and/or the like. The mail server can route, forward, and process incoming and outgoing mail messages that have been sent, relayed and/or otherwise traversing through and/or to the DIGI™.

Access to the DIGI™ mail may be achieved through a number of APIs offered by the individual Web server components and/or the operating system.

Also, a mail server may contain, communicate, generate, obtain, and/or provide program component, system, user, and/or data communications, requests, information, and/or responses.

### Mail Client

A mail client component 722 is a stored program component that is executed by a CPU 703. The mail client may be a conventional mail viewing application such as Apple Mail, Microsoft Entourage, Microsoft Outlook, Microsoft Outlook Express, Mozilla, Thunderbird, and/or the like. Mail clients may support a number of transfer protocols, such as: IMAP, Microsoft Exchange, POP3, SMTP, and/or the like. A mail client may communicate to and/or with other components in a component collection, including itself, and/or facilities of the like. Most frequently, the mail client communicates with mail servers, operating systems, other mail clients, and/or the like; e.g., it may contain, communicate, generate, obtain, and/or provide program component, system, user, and/or data communications, requests, information, and/or responses. Generally, the mail client provides a facility to compose and transmit electronic mail messages.

### Cryptographic Server

A cryptographic server component 720 is a stored program component that is executed by a CPU 703, cryptographic processor 726, cryptographic processor interface 727, cryptographic processor device 728, and/or the like. Cryptographic processor interfaces will allow for expedition of encryption and/or decryption requests by the cryptographic component; however, the cryptographic component, alternatively, may run on a conventional CPU. The cryptographic component allows for the encryption and/or decryption of provided data. The cryptographic component allows for both symmetric and asymmetric (e.g., Pretty Good Protection (PGP)) encryption and/or decryption. The cryptographic component may employ cryptographic techniques such as, but not limited to: digital certificates (e.g., X.509 authentication framework), digital signatures, dual signatures, enveloping, password access protection, public key management, and/or the like. The cryptographic component will facilitate numerous (encryption and/or decryption) security protocols such as, but not limited to: checksum, Data Encryption Standard (DES), Elliptical Curve Encryption (ECC), International Data Encryption Algorithm (IDEA), Message Digest 5 (MD5, which is a one way hash function), passwords, Rivest Cipher (RCS), Rijndael, RSA (which is an Internet encryption and authentication system that uses an algorithm developed in 1977 by Ron Rivest, Adi Shamir, and Leonard Adleman), Secure Hash Algorithm (SHA), Secure Socket Layer (SSL), Secure Hypertext Transfer Protocol (HTTPS), and/or the like. Employing such encryption security protocols, the DIGI™ may encrypt all incoming and/or outgoing communications and may serve as node within a virtual private network (VPN) with a wider communications network. The cryptographic component facilitates the process of “security authorization” whereby access to a resource is inhibited by a security protocol wherein the cryptographic component effects authorized access to the secured resource. In addition, the cryptographic component may provide unique identifiers of content, e.g., employing and MD5 hash to obtain a unique signature for a digital audio file. A cryptographic component may communicate to and/or with other components in a component collection, including itself, and/or facilities of the like. The cryptographic component supports encryption schemes allowing for the secure transmission of information across a communications network to enable the DIGI™ component to engage in secure transactions if so desired. The cryptographic component facilitates the secure accessing of resources on the DIGI™ and facilitates the access of secured resources on remote systems; i.e., it may act as a client and/or server of secured resources. Most frequently, the cryptographic component communicates with information servers, operating systems, other program components, and/or the like. The cryptographic component may contain, communicate, generate, obtain, and/or provide program component, system, user, and/or data communications, requests, and/or responses.

### The DIGI™ Database

The DIGI™ database component 719 may be embodied in a database and its stored data. The database is a stored program component, which is executed by the CPU; the stored program component portion configuring the CPU to process the stored data. The database may be a conventional, fault tolerant, relational, scalable, secure database such as Oracle or Sybase. Relational databases are an extension of a flat file. Relational databases consist of a series of related tables. The tables are interconnected via a key field. Use of the key field allows the combination of the tables by indexing against the key field; i.e., the key fields act as dimensional pivot points for combining information from various tables. Relationships generally identify links maintained between tables by matching primary keys. Primary keys represent fields that uniquely identify the rows of a table in a relational database. More precisely, they uniquely identify rows of a table on the “one” side of a one-to-many relationship.

Alternatively, the DIGI™ database may be implemented using various standard data-structures, such as an array, hash, (linked) list, struct, structured text file (e.g., XML), table, and/or the like. Such data-structures may be stored in memory and/or in (structured) files. In another alternative, an object-oriented database may be used, such as Frontier, ObjectStore, Poet, Zope, and/or the like. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the DIGI™ database is implemented as a data-structure, the use of the DIGI™ database 719 may be integrated into another component such as the DIGI™ component 735. Also, the database may be implemented as a mix of data structures, objects, and relational structures. Databases may be consolidated and/or distributed in countless variations through standard data processing techniques. Portions of databases, e.g., tables, may be exported and/or imported and thus decentralized and/or integrated.

In one embodiment, the database component 719 includes several tables 719a-n. A Users (e.g., operators and physicians) table 719a may include fields such as, but not limited to: user_id, ssn, dob, first_name, last_name, age, state, address_firstline, address_secondline, zipcode, devices_list, contact_info, contact_type, alt_contact_info, alt_contact_type, and/or the like to refer to any type of enterable data or selections discussed herein. The Users table may support and/or track multiple entity accounts. A Clients table 719b may include fields such as, but not limited to: user_id, client_id, client_ip, client_type, client_model, operating_system, os_version, app_installed_flag, and/or the like. An Apps table 719c may include fields such as, but not limited to: app_ID, app_name, app_type, OS_compatibilities_list, version, timestamp, developer_ID, and/or the like. A blood pressure parameters table 719d including, for example, measured blood pressures and other sensor outputs, such as measured_pressure, time_stamp, as well as those relating to any other variable or figure of merit described elsewhere herein and/or the like.

In one embodiment, user programs may contain various user interface primitives, which may serve to update the DIGI™ platform. Also, various accounts may require custom database tables depending upon the environments and the types of clients the DIGI™ system may need to serve. It should be noted that any unique fields may be designated as a key field throughout. In an alternative embodiment, these tables have been decentralized into their own databases and their respective database controllers (i.e., individual database controllers for each of the above tables). Employing standard data processing techniques, one may further distribute the databases over several computer systemizations and/or storage devices. Similarly, configurations of the decentralized database controllers may be varied by consolidating and/or distributing the various database components 719a-n. The DIGI™ system may be configured to keep track of various settings, inputs, and parameters via database controllers.

The DIGI™ database may communicate to and/or with other components in a component collection, including itself, and/or facilities of the like. Most frequently, the DIGI™ database communicates with the DIGI™ component, other program components, and/or the like. The database may contain, retain, and provide information regarding other nodes and data.

### The DIGI™ Components

The DIGI™ component 735 is a stored program component that is executed by a CPU. In one embodiment, the DIGI™ component incorporates any and/or all combinations of the aspects of the DIGI™ systems discussed in the previous figures. As such, the DIGI™ component affects accessing, obtaining and the provision of information, services, transactions, and/or the like across various communications networks.

The DIGI™ component may transform data collected by the device 100 or other input signals received, e.g., from a mobile device, into commands for operating the device 100.

The DIGI™ component enabling access of information between nodes may be developed by employing standard development tools and languages such as, but not limited to: Apache components, Assembly, ActiveX, binary executables, (ANSI) (Objective-) C (++), C# and/or .NET, database adapters, CGI scripts, Java, JavaScript, mapping tools, procedural and object oriented development tools, PERL, PHP, Python, shell scripts, SQL commands, web application server extensions, web development environments and libraries (e.g., Microsoft's ActiveX; Adobe AIR, FLEX & FLASH; AJAX; (D)HTML; Dojo, Java; JavaScript; jQuery(UI); MooTools; Prototype; script.aculo.us; Simple Object Access Protocol (SOAP); SWFObject; Yahoo! User Interface; and/or the like), WebObjects, and/or the like. In one embodiment, the DIGI™ server employs a cryptographic server to encrypt and decrypt communications. The DIGI™ component may communicate to and/or with other components in a component collection, including itself, and/or facilities of the like. Most frequently, the DIGI™ component communicates with the DIGI™ database, operating systems, other program components, and/or the like. The DIGI™ may contain, communicate, generate, obtain, and/or provide program component, system, user, and/or data communications, requests, and/or responses.

### Distributed DIGI™ Embodiments

The structure and/or operation of any of the DIGI™ node controller components may be combined, consolidated, and/or distributed in any number of ways to facilitate development and/or deployment. Similarly, the component collection may be combined in any number of ways to facilitate deployment and/or development. To accomplish this, one may integrate the components into a common code base or in a facility that can dynamically load the components on demand in an integrated fashion.

The component collection may be consolidated and/or distributed in countless variations through standard data processing and/or development techniques. Multiple instances of any one of the program components in the program component collection may be instantiated on a single node, and/or across numerous nodes to improve performance through load-balancing and/or data-processing techniques. Furthermore, single instances may also be distributed across multiple controllers and/or storage devices; e.g., databases. All program component instances and controllers working in concert may do so through standard data processing communication techniques.

The configuration of the DIGI™ controller will depend on the context of system deployment. Factors such as, but not limited to, the budget, capacity, location, and/or use of the underlying hardware resources may affect deployment requirements and configuration. Regardless of if the configuration results in more consolidated and/or integrated program components, results in a more distributed series of program components, and/or results in some combination between a consolidated and distributed configuration, data may be communicated, obtained, and/or provided. Instances of components consolidated into a common code base from the program component collection may communicate, obtain, and/or provide data. This may be accomplished through intra-application data processing communication techniques such as, but not limited to: data referencing (e.g., pointers), internal messaging, object instance variable communication, shared memory space, variable passing, and/or the like.

If component collection components are discrete, separate, and/or external to one another, then communicating, obtaining, and/or providing data with and/or to other component components may be accomplished through inter-application data processing communication techniques such as, but not limited to: Application Program Interfaces (API) information passage; (distributed) Component Object Model ((D)COM), (Distributed) Object Linking and Embedding ((D)OLE), and/or the like), Common Object Request Broker Architecture (CORBA), Jini local and remote application program interfaces, JavaScript Object Notation (JSON), Remote Method Invocation (RMI), SOAP, process pipes, shared files, and/or the like. Messages sent between discrete component components for inter-application communication or within memory spaces of a singular component for intra-application communication may be facilitated through the creation and parsing of a grammar. A grammar may be developed by using development tools such as lex, yacc, XML, and/or the like, which allow for grammar generation and parsing capabilities, which in turn may form the basis of communication messages within and between components.

For example, a grammar may be arranged to recognize the tokens of an HTTP post command, e.g.:
w3c-post http:// . . . Value1

where Value1is discerned as being a parameter because “http://” is part of the grammar syntax, and what follows is considered part of the post value. Similarly, with such a grammar, a variable “Value1” may be inserted into an “http://” post command and then sent. The grammar syntax itself may be presented as structured data that is interpreted and/or otherwise used to generate the parsing mechanism (e.g., a syntax description text file as processed by lex, yacc, etc.). Also, once the parsing mechanism is generated and/or instantiated, it itself may process and/or parse structured data such as, but not limited to: character (e.g., tab) delineated text, HTML, structured text streams, XML, and/or the like structured data. In another embodiment, inter-application data processing protocols themselves may have integrated and/or readily available parsers (e.g., JSON, SOAP, and/or like parsers) that may be employed to parse (e.g., communications) data. Further, the parsing grammar may be used beyond message parsing, but may also be used to parse: databases, data collections, data stores, structured data, and/or the like. Again, the desired configuration will depend upon the context, environment, and requirements of system deployment.

For example, in some implementations, the DIGI™ controller may be executing a PHP script implementing a Secure Sockets Layer (“SSL”) socket server via the information server, which listens to incoming communications on a server port to which a client may send data, e.g., data encoded in JSON format. Upon identifying an incoming communication, the PHP script may read the incoming message from the client device, parse the received JSON-encoded text data to extract information from the JSON-encoded text data into PHP script variables, and store the data (e.g., client identifying information, etc.) and/or extracted information in a relational database accessible using the Structured Query Language (“SQL”). An exemplary listing, written substantially in the form of PHP/SQL commands, to accept JSON-encoded input data from a client device via a SSL connection, parse the data to extract variables, and store the data to a database, is provided below:

```
<?PHP
header(′Content-Type: text/plain′);
// set ip address and port to listen to for incoming data
$address = ‘192.168.0.100’;
$port = 255;
// create a server-side SSL socket, listen for/accept incoming
communication
$sock = socket_create(AF_INET, SOCK_STREAM, 0);
socket_bind($sock, $address, $port) or die(‘Could not bind to address’);
socket_listen($sock);
$client = socket_accept($sock);
// read input data from client device in 1024 byte blocks until end of
message
do {
  $input = “”;
  $input = socket_read($client, 1024);
  $data .= $input;
} while($input != “”);
// parse data to extract variables
$obj = json_decode($data, true);
// store input data in a database
mysql_connect(″201.408.185.132″,$DBserver,$password);
// access database server
mysql_select(″CLIENT_DB.SQL″); // select database to append
mysql_query(“INSERT INTO UserTable (transmission)
VALUES ($data)”); // add data to UserTable table in a CLIENT
database
mysql_close(″CLIENT_DB.SQL″); // close connection to database
?>
```

Also, the following resources may be used to provide example embodiments regarding SOAP parser implementation:

http://www.xav.com/perl/site/lib/SOAP/Parser.html
http://publib.boulder.ibm.com/infocenter/tivihelp/v2r1/index.jsp?topic=/com.ibm.IBMDI. doc/referenceguide295.htm

and other parser implementations:

http://publib.boulder.ibm.com/infocenter/tivihelp/v2r1/index.jsp?topic=/com.ibm.IBMDI. doc/referenceguide259.htm

all of which are hereby expressly incorporated by reference.

In order to address various issues and advance the art, the entirety of this application (including the Cover Page, Title, Headings, Field, Background, Summary, Brief Description of the Drawings, Detailed Description, Claims, Abstract, Figures, Appendices and/or otherwise) shows by way of illustration various embodiments in which the claimed inventions may be practiced. The advantages and features of the application are of a representative sample of embodiments only, and are not exhaustive and/or exclusive. They are presented only to assist in understanding and teach the claimed principles. It should be understood that they are not representative of all disclosed embodiments. As such, certain aspects of the disclosure have not been discussed herein. That alternate embodiments may not have been presented for a specific portion of the invention or that further undescribed alternate embodiments may be available for a portion is not to be considered a disclaimer of those alternate embodiments. It will be appreciated that many of those undescribed embodiments incorporate the same principles of the invention and others are equivalent. Thus, it is to be understood that other embodiments may be utilized and functional, logical, organizational, structural and/or topological modifications may be made without departing from the scope and/or spirit of the disclosure. As such, all examples and/or embodiments are deemed to be non-limiting throughout this disclosure. Also, no inference should be drawn regarding those embodiments discussed herein relative to those not discussed herein other than it is as such for purposes of reducing space and repetition. For instance, it is to be understood that the logical and/or topological structure of any combination of any program components (a component collection), other components and/or any present feature sets as described in the figures and/or throughout are not limited to a fixed operating order and/or arrangement, but rather, any disclosed order is exemplary and all equivalents, regardless of order, are contemplated by the disclosure. Furthermore, it is to be understood that such features are not limited to serial execution, but rather, any number of threads, processes, services, servers, and/or the like that may execute asynchronously, concurrently, in parallel, simultaneously, synchronously, and/or the like are contemplated by the disclosure. As such, some of these features may be mutually contradictory, in that they cannot be simultaneously present in a single embodiment. Similarly, some features are applicable to one aspect of the invention, and inapplicable to others. In addition, the disclosure includes other inventions not presently claimed. Applicant reserves all rights in those presently unclaimed inventions including the right to claim such inventions, file additional applications, continuations, continuations in part, divisions, and/or the like thereof. As such, it should be understood that advantages, embodiments, examples, functional, features, logical, organizational, structural, topological, and/or other aspects of the disclosure are not to be considered limitations on the disclosure as defined by the claims or limitations on equivalents to the claims. It is to be understood that, depending on the particular needs and/or characteristics of a DIGI™ individual and/or enterprise user, database configuration and/or relational model, data type, data transmission and/or network framework, syntax structure, and/or the like, various embodiments of the DIGI™ may be implemented that enable a great deal of flexibility and customization.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Descriptions herein of circuitry and method steps and computer programs represent conceptual embodiments of illustrative circuitry and software embodying the principles of the disclosed embodiments. Thus the functions of the various elements shown and described herein may be provided through the use of dedicated hardware as well as hardware capable of executing software in association with appropriate software as set forth herein.

Terms to exemplify orientation, such as upper/lower, left/right, top/bottom and above/below, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein.

Similarly, it will be appreciated that the system and process flows described herein represent various processes which may be substantially represented in computer-readable media and so executed by a computer or processor, whether or not such computer or processor is explicitly shown. Moreover, the various processes can be understood as representing not only processing and/or other functions but, alternatively, as blocks of program code that carry out such processing or functions.

As examples, the Specification describes and/or illustrates aspects useful for implementing the claimed disclosure by way of various circuits or circuitry which may be illustrated as or using terms such as blocks, modules, device, system, unit, controller, and/or other circuit-type depictions. Such circuits or circuitry are used together with other elements to exemplify how certain embodiments may be carried out in the form or structures, steps, functions, operations, activities, etc. In certain embodiments, such illustrated items represent one or more computer circuitry (e.g., microcomputer or other CPU) which is understood to include memory circuitry that stores code (program to be executed as a set/sets of instructions) for performing an algorithm. The specification may also make reference to an adjective that does not connote any attribute of the structure (“first [type of structure]” and “second [type of structure]”) in which case the adjective is merely used for English-language antecedence to differentiate one such similarly-named structure from another similarly-named structure (e.g., “first circuit configured to convert . . . ” is interpreted as “circuit configured to convert . . . ”). On the other hand, specification may make reference to an adjective that is intended to connote an attribute of the structure (e.g., monitor server), in which case the adjective (e.g., monitor) modifies to refer to at least a portion of the named structure (e.g., server) is configured to have/perform that attribute (e.g., monitor server refers to at least a portion of a server that includes/performs the attribute of monitoring.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

The methods, systems, computer programs and mobile devices of the present disclosure, as described above and shown in the drawings, among other things, provide for improved methods, systems and machine readable programs for carrying out blood pressure measurement. It will be apparent to those skilled in the art that various modifications and variations can be made in the devices, methods, software programs and mobile devices of the present disclosure without departing from the spirit or scope of the disclosure. Thus, it is intended that the present disclosure include modifications and variations that are within the scope of the subject disclosure and equivalents.

What is claimed is:

1. A method of measuring blood pressure using a handheld user device including detection, processing and display circuitry, the method comprising the steps of:
guiding a user, via the processing circuitry, regarding placement of an anatomical region of interest in relation to a sensor of the handheld user device, the anatomical region of interest including an artery other than the transverse palmer arch artery;
measuring, by the sensor, a measured pressure applied to the sensor;
measuring, by the sensor, measured blood volume oscillations in the anatomical region of interest while pressure is being applied to the sensor by the anatomical region of interest;
estimating, by the processing circuitry of the device, a blood pressure value for the user from the measured pressure and the measured blood volume oscillations; and
generating a signal for presenting the blood pressure value.

2. The method of claim 1, wherein the anatomical region of interest includes a superficial artery other than the transverse palmer arch artery.

3. The method of claim 1, wherein the anatomical region of interest includes a non-superficial artery.

4. The method of claim 1, wherein the anatomical region of interest includes a digital artery other than the transverse palmer arch artery.

5. The method of claim 1, wherein the anatomical region of interest includes an artery of the hand other than the transverse palmer arch artery.

6. The method of claim 1, wherein the anatomical region of interest includes an artery of the thumb.

7. The method of claim 1, wherein the anatomical region of interest includes an inner artery of the nose.

8. The method of claim 1, wherein the anatomical region of interest includes an outer artery of the nose.

9. The method of claim 1, wherein the anatomical region of interest includes an artery of the forehead.

10. The method of claim 1, wherein the anatomical region of interest includes an artery of the scalp.

11. The method of claim 1, wherein the anatomical region of interest includes an artery of the ear.

12. The method of claim 1, wherein the anatomical region of interest includes an artery of the wrist.

13. The method of claim 12, wherein the anatomical region of interest includes the radial artery.

15. The method of claim 1, wherein the anatomical region of interest includes the brachial, axillary, subclavian or ulnar artery.

16. The method of claim 1, wherein the anatomical region of interest includes the popliteal, tibial, iliac or dorsalis artery.

17. The method of claim 1, further comprising placing a visual indicator on an exterior surface of the device, wherein the visual indicator is arranged in relation to the sensor and the sensor is integrated into the exterior surface of the device.

18. The method of claim 1, further comprising capturing an image or impression from the anatomical region of interest applied to the sensor and guiding placement of the anatomical region of interest in relation to the sensor using the image or impression.

19. The method of claim 1, further comprising:
constructing two or more oscillograms for the user from the measured pressure and the measured blood volume oscillations via the processing circuitry, wherein each oscillogram in the two or more oscillograms is constructed from measurements taken at different placements of the anatomy of interest in relation to the sensor; and
determining a particular placement for the anatomy of the user based on at least one attribute of the two or more oscillograms via the processing circuitry, and guiding the user to place the anatomy of interest at the particular placement, wherein the sensor includes a photoplethysmography (PPG) sensor and a pressure sensor.

20. The method of claim 19, wherein the pressure sensor includes a thin-filmed transducer, the PPG sensor is an array of PPG sensors including a plurality of PPG sensors and the device is configured and arranged to choose the sensor that yields a largest amplitude oscillogram.
